(19) United States
(12) Patent Application Publication
(10) Pub. No.: US 2023/0059869 A1
(43) Pub. Date: Feb. 23, 2023

(54) ORAL PHARMACEUTICAL SOLUTION OF CLOPIDOGREL

(71) Applicant: LIQMEDS WORLDWIDE LIMITED, Hayes (GB)

(72) Inventors: Jayanta Kumar MANDAL, Ahmedabad (IN); Malay PATEL, Ahmedabad (IN); Swati NAGAR, Amreli (IN)

(73) Assignee: LIQMEDS WORLDWIDE LIMITED, Hayes (GB)

(21) Appl. No.: 17/879,838

(22) Filed: Aug. 3, 2022

Related U.S. Application Data
(60) Provisional application No. 63/228,623, filed on Aug. 3, 2021.

(51) Int. Cl. A61K 31/4365; A61K 9/00; A61K 9/08; A61K 47/10; A61K 47/26

(52) U.S. Cl. CPC A61K 31/4365; A61K 9/0053; A61K 9/08; A61K 47/10; A61K 47/26

(57) ABSTRACT

Disclosed herein is an oral pharmaceutical solution, comprising: about 0.5 mg/mL to about 120 mg/mL of clopidogrel or a pharmaceutically acceptable salt thereof; about 35 mg/mL to about 200 mg/mL propylene glycol; one or more pharmaceutically acceptable excipients; and a vehicle comprising glycerin; wherein the oral pharmaceutical solution has an amount of water of less than or equal to 5% w/w and an amount of ethanol less than or equal to 6% w/w. Oral pharmaceutical solution disclosed herein have a clopidogrel content of 100±10% when stored for about 24 months at 25° C.±2° C. and 60±5% relative humidity.

### RELATED APPLICATION

This application claims priority to U.S. Provisional Patent Application No. 63/228,623, filed on Aug. 3, 2021, the subject matter of which is incorporated by reference in its entirety.

### FIELD

This disclosure relates to an oral pharmaceutical solution comprising clopidogrel or a pharmaceutically acceptable salt thereof.

### BACKGROUND

On Nov. 17, 1997, the U.S. Food and Drug Administration (“FDA”) approved PLAVIX® (clopidogrel bisulfate) tablets, for oral use. Clopidogrel is a thienopyridine class inhibitor of P2Y₁₂ ADP platelet receptors. According to the FDA-approved prescribing information as of May 17, 2019 (“PLAVIX® PI”), PLAVIX®, in combination with aspirin, is indicated generally for the use in patients with acute coronary syndrome. PLAVIX® also is indicated generally for use in patents with recent myocardial infarction, recent stroke, or established peripheral arterial disease. And pediatric patients with certain types of heart disease at risk of thrombotic events may benefit from the administration of clopidogrel (in combination with aspirin). See, e.g., Li.

Clopidogrel bisulfate (alternatively known as clopidogrel hydrogen sulfate) is an enantiomeric substance with a chemical name methyl (+)-(S)-α-(2-chlorophenyl)-6,7-dihydrothieno[3,2-c]pyridin-5(4H)-acetate sulfate (1:1). The empirical formula of clopidogrel bisulfate is C₁₆H₁₆ClNO₂S.H₂SO₄ and its molecular weight is 419.9. The structural formula is as follows:

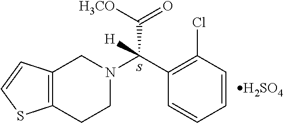

Clopidogrel bisulfate is practically insoluble in water at pH values between about 2 and about 7, but freely soluble at pH 1.

PLAVIX® for oral administration is provided as either pink, round, biconvex, debossed, film-coated tablets containing 97.875 mg of clopidogrel bisulfate (the molar equivalent of 75 mg of clopidogrel) or pink, oblong, debossed film-coated tablets containing 391.5 mg of clopidogrel bisulfate which is the mole equivalent of 300 mg of clopidogrel base. Each tablet contains hydrogenated castor oil, hydroxypropyl cellulose, mannitol, microcrystalline cellulose, and polyethylene glycol 6000 as inactive ingredients. The pink film coating contains ferric oxide, hypromellose 2910, lactose monohydrate, titanium dioxide, and triacetin. The tablets are polished with Carnauba wax. PLAVIX® is administered as an oral tablet at a recommended dose of 75 mg once daily following an initial dose of 300 mg.

Bouloumie generally discloses a clopidogrel-containing freeze-dried composition containing, among other things, mannitol and alanine. However, at the time of reconstitution of such lyophilisate in solvent, one often encounters a problem of self-aggregation of clopidogrel.

Mosher discloses aqueous compositions containing, among other things, clopidogrel bisulfate, a cyclodextrin (e.g., a sulfoalkyl ether cyclodextrin or hydroxypropyl-β-cyclodextrin), and optionally, a surfactant (e.g., polyethylene glycol-15-hydroxstearate (SOLUTOL®) or polysorbate 80 (TWEEN 80®)). The added cyclodextrin reportedly improves clopidogrel stability and solubility.

Aubert discloses a composition of clopidogrel or a pharmaceutically acceptable salt thereof for parenteral administration as an injectable solution. However, the pH of such solutions is low to accommodate the solubility profile of clopidogrel. Resulting solutions have a pH below 2, making the resulting injection very painful which can hinder patient compliance. Moreover, this type of formulation containing a salt of clopidogrel in isotonic solution in a solvent is difficult to use.

Sanofi-Synthelabo discloses injectable aqueous solutions containing a salt of clopidogrel, pluronic F68, a basic pH modifier, and Solutol HS 15. The patent further discloses lyophilized formulations containing these ingredients and kits containing such lyophilized compositions in two parts. However, according to the patent, it is necessary to achieve a lyophilizate since the salts of clopidogrel are unstable in aqueous solution and leads to clopidogrel degradation.

Cadila discloses a ready-to-use aqueous injectable formulation containing, clopidogrel besylate, a solubilizer (e.g., polyethylene glycol 660 12-hydroxystearate (SOLUTOL HS 15®) or polysorbate 80 (TWEEN 80®)), a stabilizing agent (e.g., EDTA), and a buffering agent (e.g., phosphate buffer).

Skillman describes an extemporaneously prepared clopidogrel oral suspension prepared from ground clopidogrel bisulfate tablets USP suspended in a vehicle of ORA-PLUS® and ORA-SWEET®. Skillman states that extemporaneously compounded suspensions of clopidogrel 5 mg/mL in a 1:1 mixture of ORA-PLUS® and ORA-SWEET® were stable for at least 60 days when stored at room temperature. Engels explains that suspensions often require on-demand compounding and result in increased risks for error during compounding the suspensions and dosing errors. Thus, extemporaneously prepared suspensions may be disadvantageous at least because of transfer errors, sediment suspension, and long-term stability.

Rosemont discloses generally a clopidogrel-containing composition having a high amount of ethanol (10% v/v). As stated above, clopidogrel is used in combination with aspirin. Aspirin in combination with ethanol is contraindicated because of the increased damage to gastrointestinal mucosa and bleeding time due to additive effects of aspirin and ethanol. See Bayer Information at 6. Thus, Rosemont's clopidogrel-containing composition is pharmaceutically unacceptable at least because clopidogrel is used in combination with aspirin and aspirin is contraindicated with ethanol. Additionally, Rosemont's clopidogrel-containing solution is pharmaceutically unacceptable to certain patient populations (e.g., pediatric patients or patients lacking the ability to metabolize ethanol) because of the high amount of ethanol. See, e.g., Weathermon.

A pharmaceutically acceptable oral clopidogrel-containing solution disclosed herein provides advantages over the prior dosage forms. For instance, patients who have difficulty swallowing solid oral dosage forms, including the pediatric and geriatric populations, may find particular advantage in an oral pharmaceutical solution. Dosing consistency, individualization of dosing, and patient compliance may all be achieved by an oral pharmaceutical solution formulation. Errors resulting from on-demand compounding of suspensions may be eliminated. Further, a pharmaceutically acceptable clopidogrel-containing solution disclosed herein reduces potentially undesirable side-effects (due to clopidogrel-aspirin co-administration), as well as the undesirable adverse effects arising from co-administering ethanol and aspirin. For instance, an oral liquid solution reduces potential of gastric bleeding of a solid dosage form. See Rao at 19-20. Additionally, an oral liquid solution substantially free of ethanol reduces potential gastric bleeding caused by aspirin and exacerbated by concurrent ingestion of ethanol. See Bayer Information at 6.

### SUMMARY

Disclosed herein is an oral pharmaceutical solution, comprising: about 0.5 mg/mL to about 120 mg/mL of clopidogrel or a pharmaceutically acceptable salt thereof, about 35 mg/mL to about 200 mg/mL propylene glycol; one or more pharmaceutically acceptable excipients; and a vehicle comprising glycerin; wherein the oral pharmaceutical solution has an amount of water of less than or equal to 5% w/w and an amount of ethanol less than or equal to 6% w/w. Oral pharmaceutical solutions disclosed herein have a clopidogrel content of 100±10% when stored for about 24 months at 25° C.±2° C. and 60±5% relative humidity.

### Definitions

As used herein, the singular forms “a,” “an,” “the,” “one or more,” and “at least one” and similar expressions in the context of the oral pharmaceutical solutions disclosed herein are to be construed to cover both the singular and the plural, unless otherwise indicated herein or clearly contradicted by context.

Unless defined otherwise, all technical and scientific terms used herein have the same meanings as commonly understood by one of ordinary skill in the art.

The expression “% w/w,” as used herein, refers to the percent weight of an identified component based on the total weight of the oral pharmaceutical solution.

The expression “% v/v,” as used herein, refers to the percent volume of an identified component based on the total volume of the oral pharmaceutical solution.

The term “about” as used herein embodies standard error associated with a physicochemical observable. As used herein, the term “about” means a slight variation of the value specified, for example, within 10% of the value specified. A stated amount for a compositional ingredient that is not preceded by the term “about” does not mean that there is no variance for the stated term, as one of ordinary skill would understand that there may be the possibility of a degree of variability generally associated with experimental error.

The expressions “optional” or “optionally” means that the subsequently described event or circumstance may or may not occur, and that the description includes instances where the event occurs and instances where it does not.

The expression “pharmaceutically acceptable”, as used herein refers to an excipient having compatibility with the other ingredients of the formulation and not deleterious to the recipient thereof.

The expression “quantity sufficient” or “q.s.,” as used herein refers to the amount of a substance required to meet a certain parameter, for instance as a makeup volume of solvent or vehicle required to provide a solution of a given volume.

The expression “dosage dispensing unit” as used herein is intended to mean a container that is known in the medical arts to be useful for storing liquid pharmaceuticals prior to administration, and from which portions of the contents, for example specific doses to be administered to a patient, can be withdrawn. Examples of such dosage dispensing units include, but are not limited to, bottles, ampoules, vials, syringes, etc.; wherein a bottle comprises a removable cap, for example a screw cap or snap-on cap; or a septum; or a metered dispensing cap; wherein a bottle is with a septum adapted to allow removal of liquid, wherein the septum allows removal of liquid by use of a syringe (an oral syringe).

The expression “direct dosing device” as used herein is intended to mean a device or container that is used to administer its contents directly to a patient. Examples of such direct dosing devices include, but are not limited to, syringes, for example oral syringes, cups for example dosing cups, an ampoule, a vial, a syringe, spoons for example dosage spoons, teaspoons and tablespoons, a dropper or dropper assembly, a pipette, a measuring device with graduations, or any device customarily used for, or capable of being used to dispense a liquid medicament to a patient. Such devices, including dosage cups, dosage spoons, droppers and dropper assemblies, oral syringes, bottles with fitments, and total dispensing systems are sold by Comar, Voorhees, N.J. Examples of the foregoing dosage cups, dosage spoons, droppers and dropper assemblies, oral syringes, and total dispensing systems can be found on the Comar website at www.comar.com, the O.Berk website at www.oberk.com, and the Drug Plastic website at www.drugplastics.com.

### DETAILED DESCRIPTION

Disclosed herein is an oral pharmaceutical solution, comprising: about 0.5 mg/mL to about 120 mg/mL of clopidogrel or a pharmaceutically acceptable salt thereof, about 35 mg/mL to about 200 mg/mL propylene glycol; one or more pharmaceutically acceptable excipients; and a vehicle comprising glycerin; wherein the oral pharmaceutical solution has an amount of water of less than or equal to 5% w/w and an amount of ethanol less than or equal to 6% w/w. Oral pharmaceutical solution disclosed herein have a clopidogrel content of 100±10% when stored for about 24 months at 25° C.±2° C. and 60±5% relative humidity.

In one aspect, the amount of clopidogrel or a pharmaceutically acceptable salt thereof, based on clopidogrel, ranges from about 0.5 mg/mL to about 120 mg/mL, and all values in between, for example, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, and about 110 mg/mL.

In other aspects, the amount of clopidogrel or a pharmaceutically acceptable salt thereof, based on clopidogrel may be within a range from about 5 mg/mL to about 35 mg/mL. Alternatively, the amount of clopidogrel or a pharmaceutically acceptable salt thereof, based on clopidogrel may be within a range from about 10 mg/mL to about 25 mg/mL, from about 5 mg/mL to 35 mg/mL, from about 10 mg/mL to about 25 mg/mL, or from about 13 mg/mL to about 17 mg/mL.

Pharmaceutically acceptable salts of clopidogrel include, but are not limited to clopidogrel bisulfate, clopidogrel besylate, clopidogrel maleate, clopidogrel benzoate, clopidogrel tosylate, clopidogrel succinate, clopidogrel salicylate, clopidogrel acetate, and clopidogrel hydrochloride, as well as other pharmaceutically acceptable salts described, for example, by Berge.

In one aspect, a pharmaceutically acceptable salt relates to clopidogrel bisulfate. For ease of reference, the reported molecular weight of clopidogrel bisulfate is 419.9 g/mol (“CBMW”), while the reported molecular weight of clopidogrel is 321.8 g/mol (“CMW”). One may readily convert an amount of clopidogrel bisulfate to an amount of clopidogrel by multiplying the amount of clopidogrel bisulfate by a factor that corresponds to the ratio of the respective molecular weights of clopidogrel and clopidogrel bisulfate (viz., CMW/CBMW=321.8/419.9≈0.766).

Oral pharmaceutical solutions disclosed herein result in a clopidogrel stability providing a shelf-life stability of at least 2 years. Stability may be ascertained by measure of the clopidogrel remaining in solution relative to an initial concentration. In other words, the oral pharmaceutical solutions disclosed herein exhibit prolonged stability when stored for at least about 24-months at 25±2° C. and 60±5% relative humidity, and thus, room temperature stable products when stored for about 1-month or more, about 3-months or more, about 6-months or more, about 9-months or more, about 12-months or more, about 18-months or more, or about 24-months or more (e.g., 36-months, 48-months, etc.). For example, an oral pharmaceutical solution disclosed herein has a clopidogrel content of 100±10% clopidogrel labeled content when stored for about 24-months (or more) at 25±2° C. and 60±5% relative humidity.

Alternatively, stability of oral pharmaceutical solutions may be assessed by observing the amount of impurities present in the solution relative to an initial mixture. Impurities may be introduced to the oral pharmaceutical solution by degradation of clopidogrel (viz., degradation impurities).

Oral pharmaceutical solutions disclosed herein result in a reduced amount of Clopidogrel Impurity A (viz., Impurity A, CAS No. 144457-28-3, clopidogrel carboxylic acid or (S)-2-(2-chlorophenyl)-2-(6,7-dihydrothieno[3,2-c]pyridin-5(4H)-yl) acetic acid). Impurity A is a hydrolysis degradant of clopidogrel that generally increases with time depending on the stability of the solution. See, e.g., Reist at 1408. Impurity A has the following structure:

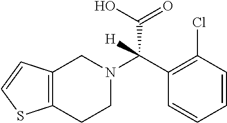 [SMILES: O=C(O)[C@H](c1ccccc1Cl)N1CCc2sccc2C1]

In certain aspects, an oral pharmaceutical solution disclosed herein has an amount of Impurity A of not more than about 1.2% w/w when stored for at least about 24-months at 25±2° C. and 60±5% relative humidity.

Oral pharmaceutical solutions disclosed herein result in a reduced amount of Impurity C (CAS No. 120202-69-9). Impurity C is an enantiomeric impurity that may be present in the initial preparation as a synthetic impurity, but also may increase due to clopidogrel degradation. See, e.g., Reist at 1408. Impurity C has the chemical name (R)-Methyl (2-chlorophenyl)(6,7-dihydro-4H-thieno[3,2-c]pyridin-5-yl)acetate, and is alternatively known as (R)-clopidogrel (or SR 25989). The chemical structure of Impurity C is shown below.

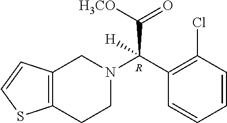

Impurity C is devoid of antithrombotic activity and can evoke convulsions at high doses. See Reist at 1405; see also Badorc at columns 9-10.

An oral pharmaceutical solution contemplated herein has an amount of Impurity C of not more than about 1.5% w/w when stored for at least about 24-months at 25±2° C. and 60±5% relative humidity.

Oral pharmaceutical solutions disclosed herein result in a reduced amount of Clopidogrel Impurity F (viz., Impurity F, CAS No. 141109-20-8). Impurity F is known chemically as (S)-Methyl 2-(2-chlorophenyl)-2-((2-(thiophen-2-yl)ethyl)amino)acetate and a chemical structure as shown below.

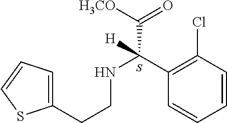

An oral pharmaceutical solution contemplated herein has an amount of Impurity F of not more than about 0.2% w/w when stored for at least about 24-months at 25±2° C. and 60±5% relative humidity.

Oral pharmaceutical solutions disclosed herein comprise at least one pharmaceutically acceptable excipient, which may include a sweetener agent, an antioxidant, optionally a co-solvent, a flavorant, or a combination thereof.

The amount of a sweetener agent, if present, ranges from about 0 mg/mL to about 10 mg/mL, and all values in between, for example, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, about 2.0 mg/mL, about 2.1 mg/mL, about 2.2 mg/mL, about 2.3 mg/mL, about 2.4 mg/mL, about 2.5 mg/mL, about 2.6 mg/mL, about 2.7 mg/mL, about 2.8 mg/mL, about 2.9 mg/mL, about 3.0 mg/mL, about 3.1 mg/mL, about 3.2 mg/mL, about 3.3 mg/mL, about 3.4 mg/mL, about 3.5 mg/mL, about 3.6 mg/mL, about 3.7 mg/mL, about 3.8 mg/mL, about 3.9 mg/mL, about 4.0 mg/mL, about 4.1 mg/mL, about 4.2 mg/mL, about 4.3 mg/mL, about 4.4 mg/mL, about 4.5 mg/mL, about 4.6 mg/mL, about 4.7 mg/mL, about 4.8 mg/mL, about 4.9 mg/mL, about 5.0 mg/mL, about 5.1 mg/mL, about 5.2 mg/mL, about 5.3 mg/mL, about 5.4 mg/mL, about 5.5 mg/mL, about 5.6 mg/mL, about 5.7 mg/mL, about 5.8 mg/mL, about 5.9 mg/mL, about 6.0 mg/mL, about 6.1 mg/mL, about 6.2 mg/mL, about 6.3 mg/mL, about 6.4 mg/mL, about 6.5 mg/mL, about 6.6 mg/mL, about 6.7 mg/mL, about 6.8 mg/mL, about 6.9 mg/mL, about 7.0 mg/mL, about 7.1 mg/mL, about 7.2 mg/mL, about 7.3 mg/mL, about 7.4 mg/mL, about 7.5 mg/mL, about 7.6 mg/mL, about 7.7 mg/mL, about 7.8 mg/mL, about 7.9 mg/mL, about 8.0 mg/mL, about 8.1 mg/mL, about 8.2 mg/mL, about 8.3 mg/mL, about 8.4 mg/mL, about 8.5 mg/mL, about 8.6 mg/mL, about 8.7 mg/mL, about 8.8 mg/mL, about 8.9 mg/mL, about 9.0 mg/mL, about 9.1 mg/mL, about 9.2 mg/mL, about 9.3 mg/mL, about 9.4 mg/mL, about 9.5 mg/mL, about 9.6 mg/mL, about 9.7 mg/mL, about 9.8 mg/mL, and about 9.9 mg/mL.

Examples of sweetener agents include, but are not limited to acesulfame potassium, alitame, aspartame, compressible sugar, confectioner's sugar, dextrose, erythritol, fructose, glycerin, glycine, inulin, isomalt, lactitol, liquid glucose, maltitol, maltitol solution, a maltitol oligomer, maltose, mannitol, neohesperidin dihydrochalcone, neotame, saccharin, saccharin sodium, sodium cyclamate, sorbitol, sucralose, sucrose, tagatose, thaumatin, trehalose, and xylitol.

In one aspect, the sweetener agent comprises sucralose. In one aspect, the sweetener agent comprises sucralose in an amount of from about 0.5 mg/mL to about 2.0 mg/mL, and all values in between, for example, about 0.6 mg/mL, about 0.7 mg/mL, about 0.8 mg/mL, about 0.9 mg/mL, about 1.0 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, and about 1.9 mg/mL.

An antioxidant, if present, ranges from about 0.01 mg/mL to about 0.2 mg/mL and all values in between, for example, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, about 0.11 mg/mL, about 0.12 mg/mL, about 0.13 mg/mL, about 0.14 mg/mL, about 0.15 mg/mL, about 0.16 mg/mL, about 0.17 mg/mL, about 0.18 mg/mL, and about 0.19 mg/mL.

An antioxidant may also be included in the oral pharmaceutical solutions disclosed herein in an amount of more than 0.2 mg/mL (e.g. from about 0.5 mg/mL to about 5.0 mg/mL and all values in between) if it does not negatively affect the quality of the product (for example increased amounts of impurities mentioned herein or any unspecified impurity).

Examples of antioxidants include, but are not limited to ascorbyl palmitate, butylated hydroxyanisole, butylated hydroxytoluene, erythorbic acid, ethyl oleate, methionine, monothioglycerol, propyl gallate, sodium ascorbate, thymol, tocopherol (e.g., alpha tocopherol), vitamin E, and vitamin E polyethylene glycol succinate and the like or any antioxidant which is pharmaceutically acceptable and does not affect the basic and novel characteristic of the oral pharmaceutical solution disclosed herein, and which is known to be useful in the preparation of oral solution dosage forms.

In one aspect, the antioxidant comprises butylated hydroxyanisole in an amount of about 0.01 mg/mL to 0.2 mg/mL and all values in between, including, for example, 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.11 mg/mL, 0.12 mg/mL, 0.13 mg/mL, 0.14 mg/mL, 0.15 mg/mL, 0.16 mg/mL, 0.17 mg/mL, 0.18 mg/mL, and 0.19 mg/mL. In another aspect, the antioxidant comprises butylated hydroxyanisole in an amount of about 0.05 mg/mL to about 0.15 mg/mL. In yet another aspect, the antioxidant comprises butylated hydroxyanisole in an amount of about 0.1 mg/mL, where butylated hydroxyanisole exhibits antimicrobial activity.

One or more cosolvents may also be present in the oral pharmaceutical solutions disclosed herein, for purpose of maintaining dissolved components, stability, and resistance to thermal excursions. Examples of such cosolvents include, but are not limited to, an alcohol (e.g., ethanol), a glycol (e.g., propylene glycol, ethylene glycol, glycerol behenate, and a polyethylene glycol), a sugar alcohol (e.g., maltitol, sorbitol, xylitol, erythritol, etc.) and the like or any solvent which is pharmaceutically acceptable and does not affect the basic and novel characteristic of the oral pharmaceutical solution disclosed herein, and which is known to be useful in the preparation of oral solution dosage forms.

Ethanol, if present, ranges from about 0.1% v/v to about 9.5% v/v, based on the total volume of the solution, and all values in between, for example, 0.1% v/v, 0.2% v/v, 0.3% v/v, 0.4% v/v, 0.5% v/v, about 0.6% v/v, about 0.7% v/v, about 0.8% v/v, about 0.9% v/v, about 1.0% v/v about 1.1% v/v, about 1.2% v/v, about 1.3% v/v, about 1.4% v/v, about 1.5% v/v, about 1.6% v/v, about 1.7% v/v, about 1.8% v/v, about 1.9% v/v, about 2.0% v/v, about 2.1% v/v, about 2.2% v/v, about 2.3% v/v, about 2.4% v/v, about 2.5% v/v, about 2.6% v/v, about 2.7% v/v, about 2.8% v/v, about 2.9% v/v, about 3.0% v/v, about 3.1% v/v, about 3.2% v/v, about 3.3% v/v, about 3.4% v/v, about 3.5% v/v, about 3.6% v/v, about 3.7% v/v, about 3.8% v/v, about 3.9% v/v, about 4.0% v/v, about 4.1% v/v, about 4.2% v/v, about 4.3% v/v, about 4.4% v/v, about 4.5% v/v, about 4.6% v/v, about 4.7% v/v, about 4.8% v/v, about 4.9% v/v, about 5.0% v/v, about 5.1% v/v, about 5.2% v/v, about 5.3% v/v, about 5.4% v/v, about 5.5% v/v, about 5.6% v/v, about 5.7% v/v, about 5.8% v/v, about 5.9% v/v, about 6.0% v/v, about 6.1% v/v, about 6.2% v/v, about 6.3% v/v, about 6.4% v/v, about 6.5% v/v, about 6.6% v/v, about 6.7% v/v, 6.8% v/v, about 6.9% v/v, about 7.0% v/v, about 7.1% v/v, about 7.2% v/v, about 7.3% v/v, about 7.4% v/v, about 7.5% v/v, about 7.6% v/v, about 7.7% v/v, about 7.8% v/v, about 7.9% v/v, about 8.0% v/v, about 8.1% v/v, about 8.2% v/v, about 8.3% v/v, about 8.4% v/v, about 8.5% v/v, about 8.6% v/v, about 8.7% v/v, about 8.8% v/v, about 8.9% v/v, about 9.0% v/v, about 9.1% v/v, about 9.2% v/v, about 9.3% v/v, about 9.4% v/v, about 9.5% v/v, about 9.6% v/v, about 9.7% v/v, about 9.8% v/v, and about 9.9% v/v. In one aspect, oral pharmaceutical solutions disclosed herein have an amount of ethanol of not more than about 4% v/v. In another aspect, oral pharmaceutical solutions disclosed herein have an amount of ethanol of from about 2% v/v to about 4% v/v. In certain aspects, oral pharmaceutical solutions disclosed herein have no added ethanol such that the ethanol content is about 0% v/v.

Propylene glycol, if present as a cosolvent, ranges from about 35 mg/mL to about 200 mg/mL, and all values in between, for example about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, and about 150 mg/mL, about 160 mg/mL, about 170 mg/mL, about 175 mg/mL, about 180 mg/mL, about 185 mg/mL, about 190 mg/mL, and about 195 mg/mL. In one aspect, oral pharmaceutical solutions disclosed herein have an amount of propylene glycol in an amount of from about 35 mg/mL to about 200 mg/mL. In another aspect, oral pharmaceutical solutions disclosed herein have an amount of propylene glycol in an amount of from about 70 mg/mL to about 200 mg/mL. In yet another aspect, oral pharmaceutical solutions disclosed herein have an amount of propylene glycol in an amount of from about 50 mg/mL to about 200 mg/mL or from about 50 mg/mL to about 100 mg/mL. In certain aspects, oral pharmaceutical solutions disclosed herein have no added propylene glycol such that the propylene glycol content is about 0% v/v.

As explained herein, an ethanol-free composition comprising clopidogrel bisulfate, propylene glycol (30 mg/mL), and glycerin resulted in a hazy solution (viz., a suspension). An ethanol-free composition comprising clopidogrel bisulfate, propylene glycol (70 mg/mL), and glycerin resulted in a clear solution. An ethanol-free composition comprising clopidogrel bisulfate, propylene glycol (200 mg/mL), and glycerin resulted in a clear solution. Thus, oral pharmaceutical solutions disclosed herein comprise an amount of propylene glycol of: (i) about 35 mg/mL to about 200 mg/mL, (ii) about 50 mg/mL to about 200 mg/mL, (iii) about 70 mg/mL to about 200 mg/mL, (iv) about 50 mg/mL to about 200 mg/mL, (v) about 50 mg/mL to about 100 mg/mL, and, in certain aspects, no added propylene glycol.

In a certain aspect, the oral pharmaceutical solution comprises an amount of ethanol of not more than 6% v/v (including an ethanol-free solution) and an amount of propylene glycol that ranges from about 35 mg/mL to about 200 mg/mL, and all values in between, including for example an amount of propylene glycol of about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, and about 195 mg/mL.

In a certain aspect, the oral pharmaceutical solution comprises an amount of ethanol of not more than about 4% v/v (including an ethanol-free solution or a solution having about 2% v/v to about 4% v/v) and an amount of propylene glycol that ranges from about 35 mg/mL to about 200 mg/mL, and all values in between, including for example an amount of propylene glycol of about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, and about 195 mg/mL.

Examples of flavorants include, but are not limited to, cherry, orange, banana, strawberry or other acceptable fruit flavors, or mixtures of cherry, orange, and other acceptable fruit flavors. Additional examples of flavorants include, but are not limited to, cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil and the like or any combinations thereof. Further examples of flavorants include, but are not limited to, vanilla, citrus oil, including lemon, orange, grape, lime and grapefruit, and fruit essences, including apple, banana, pear, peach, strawberry, raspberry, cherry, plums, pineapple, apricot, peppermint, Tutti Frutti flavor, Frozen Peppermint flavor, mixed berry, and so forth and the like or any combinations thereof. Solid forms, such as spray dried forms of flavorants, may also be useful in the liquid dosage forms disclosed herein.

A flavorant, if present, ranges from about 0.01 mg/mL to about 5.0 mg/mL, and all values in between, for example, about 0.5 mg/mL, about 1.0 mg/mL, about 1.5 mg/mL, about 2.0 mg/mL, about 2.5 mg/mL, about 3.0 mg/mL, about 3.5 mg/mL, about 4.0 mg/mL, about 4.5 mg/mL, and about 5.0 mg/mL.

The vehicle comprising glycerin may be present in a sufficient (viz., q.s.) amount to achieve a stated ingredient concentration, e.g., clopidogrel concentration. A vehicle comprising glycerin comprises an amount of glycerin that ranges from about 73% w/w to about 99% w/w, and all values in between, for example, about 74% w/w, about 75% w/w, about 76% w/w, about 77% w/w, about 78% w/w, about 79% w/w, about 80% w/w, about 81% w/w, about 82% w/w, about 83% w/w, about 84% w/w, about 85% w/w, about 86% w/w, about 87% w/w, about 88% w/w, about 89% w/w, about 90% w/w, about 91% w/w, about 92% w/w, about 93% w/w, about 94% w/w, about 95% w/w, about 96% w/w, about 97% w/w, and about 98% w/w.

The oral pharmaceutical solutions disclosed herein are non-aqueous solutions, which may be substantially free of water. Therefore, any organic solvent which is known to be useful in preparing non-aqueous oral solutions in pharmaceutical field may also be used in place of glycerin or in combination with glycerin as a vehicle provided it does not affect the basic and novel characteristic of the oral pharmaceutical solution disclosed herein.

In another aspect, the disclosure relates to solution for oral delivery. Particularly, the oral pharmaceutical solution comprises clopidogrel or a pharmaceutically acceptable salt thereof, one or more pharmaceutically acceptable excipients, and a vehicle comprising glycerin.

In another aspect, the disclosure relates to process for preparing an oral pharmaceutical solution for oral delivery. Particularly, a process for the preparation of an oral pharmaceutical solution comprising clopidogrel or a pharmaceutically acceptable salts thereof, a pharmaceutically acceptable excipient, and a vehicle comprising glycerin.

In another aspect, the disclosure relates to an oral pharmaceutical solution comprising 0.5 mg/mL to 120 mg/ml of clopidogrel or pharmaceutically acceptable salts thereof, a vehicle comprising glycerin in an amount of from about 0.9 g/mL to about 1.25 g/mL; and a cosolvent comprising propylene glycol, ethanol, or a combination thereof.

In another aspect, the disclosure relates to an oral pharmaceutical solution comprising about 5 mg/mL to about 35 mg/mL clopidogrel or a pharmaceutically acceptable salt thereof (e.g., about 15 mg/mL), about 0.05 mg/mL to 0.5 mg/mL of an antioxidant. In an aspect, the antioxidant is butylated hydroxyanisole.

### First Aspect

Another aspect relates to an oral pharmaceutical solution comprising about 13 mg/mL to about 17 mg/mL clopidogrel or a pharmaceutically acceptable salt thereof; about 35 mg/mL to about 200 mg/mL propylene glycol; about 0 mg/mL to about 75 mg/mL ethanol; one or more pharmaceutically acceptable excipients; and a vehicle comprising glycerin; wherein the ethanol content is not more than about 9.5% v/v (or about 6% w/w); wherein the amount of clopidogrel is based on the mass amount of clopidogrel per milliliter of solution.

Another aspect relates to an oral pharmaceutical solution comprising about 1.0% w/w to about 1.4% w/w clopidogrel or a pharmaceutically acceptable salt thereof, and all values in between, e.g., about 1.2% w/w; about 2.8% w/w to about 16% w/w propylene glycol, and all values in between, e.g., about 4% w/w, about 5% w/w, about 6% w/w, about 7% w/w, about 8% w/w, about 9% w/w, about 10% w/w, about 11% w/w, about 12% w/w, about 13% w/w, about 14% w/w, and about 15% w/w; about 0% w/w to about 6% w/w ethanol, and all values in between, e.g., about 1% w/w, about 2% w/w, about 3% w/w, about 4% w/w, about 5% w/w; one or more pharmaceutically acceptable excipients; and a vehicle comprising glycerin in an amount of about 76% w/w to about 96% w/w, and all values in between, e.g., about 78% w/w, about 80% w/w, about 82% w/w, about 84% w/w, about 86% w/w, about 88% w/w, about 90% w/w, about 92% w/w, and about 94% w/w; wherein the amount of clopidogrel is based on the mass amount of clopidogrel per mass amount of solution.

Yet another aspect relates to an oral pharmaceutical solution comprising about 1.0% w/w to about 1.4% w/w clopidogrel or a pharmaceutically acceptable salt thereof, about 2.8% w/w to about 16% w/w propylene glycol; about 0% w/w to about 6% w/w ethanol; about 0.005% w/w to about 0.01% w/w of an antioxidant; about 0.008% w/w to about 0.12% w/w of a sweetener agent; about 0.01% w/w to about 0.08% w/w of a flavoring agent; and a vehicle comprising glycerin in an amount of about 76% w/w to about 96% w/w; wherein the amount of clopidogrel is based on the mass amount of clopidogrel per mass amount of solution.

Yet another aspect relates to an oral pharmaceutical solution comprising about 1.0% w/w to about 1.4% w/w clopidogrel or a pharmaceutically acceptable salt thereof, about 2.8% w/w to about 16% w/w propylene glycol; about 0% w/w to about 6% w/w ethanol; about 0.005% w/w to about 0.01% w/w of an antioxidant (e.g., butylated hydroxyanisole); about 0.008% w/w to about 0.12% w/w of a sweetener agent (e.g., sucralose); about 0.01% w/w to about 0.08% w/w of a flavoring agent; and a vehicle comprising glycerin in an amount of about 76% w/w to about 96% w/w; wherein the amount of clopidogrel is based on the mass amount of clopidogrel per mass amount of solution.

A further aspect relates to an oral pharmaceutical solution comprising about 1.4% w/w to about 1.8% w/w clopidogrel bisulfate; about 2.8% w/w to about 16% w/w propylene glycol; about 0% w/w to about 6% w/w ethanol; about 0.005% w/w to about 0.01% w/w of an antioxidant; about 0.01% w/w to about 0.12% w/w of a sweetener agent; about 0.01% w/w to about 0.08% w/w of a flavoring agent; and a vehicle comprising glycerin in an amount of about 76% w/w to about 96% w/w; wherein the amount of clopidogrel bisulfate is based on the mass amount of clopidogrel per mass amount of solution.

A further aspect relates to an oral pharmaceutical solution comprising about 1.4% w/w to about 1.8% w/w clopidogrel bisulfate; about 2.8% w/w to about 16% w/w propylene glycol; about 0% w/w to about 6% w/w ethanol; about 0.005% w/w to about 0.01% w/w of an antioxidant (e.g., butylated hydroxyanisole); about 0.01% w/w to about 0.12% w/w of a sweetener agent (e.g., sucralose); about 0.01% w/w to about 0.08% w/w of a flavoring agent; and a vehicle comprising glycerin in an amount of about 76% w/w to about 96% w/w; wherein the amount of clopidogrel bisulfate is based on the mass amount of clopidogrel per mass amount of solution.

### Second Aspect

Another aspect relates to an oral pharmaceutical solution comprising about 13 mg/mL to about 17 mg/mL clopidogrel or a pharmaceutically acceptable salt thereof; about 35 mg/mL to about 200 mg/mL propylene glycol; about 16 mg/mL to about 33.2 mg/mL ethanol; one or more pharmaceutically acceptable excipients; and a vehicle comprising glycerin; wherein the ethanol content is not more than about 4% v/v (or about 2.7% w/w); wherein the amount of clopidogrel is based on the mass amount of clopidogrel per milliliter of solution.

Another aspect relates to an oral pharmaceutical solution comprising about 1.0% w/w to about 1.4% w/w clopidogrel or a pharmaceutically acceptable salt thereof, and all values in between, e.g., about 1.2% w/w; about 2.8% w/w to about 16.3% w/w propylene glycol, and all values in between, e.g., about 4% w/w, about 5% w/w, about 6% w/w, about 7% w/w, about 8% w/w, about 9% w/w, about 10% w/w, about 11% w/w, about 12% w/w, about 13% w/w, about 14% w/w, and about 15% w/w; about 0% w/w to about 2.7% w/w ethanol; one or more pharmaceutically acceptable excipients; and a vehicle comprising glycerin in an amount of about 79% w/w to about 96% w/w, and all values in between, e.g., about 80% w/w, about 82% w/w, about 84% w/w, about 86% w/w, about 88% w/w, about 90% w/w, about 92% w/w, and about 94% w/w; wherein the amount of clopidogrel is based on the mass amount of clopidogrel per mass amount of solution.

Yet another aspect relates to an oral pharmaceutical solution comprising about 1.0% w/w to about 1.4% w/w clopidogrel or a pharmaceutically acceptable salt thereof, about 2.8% w/w to about 16.3% w/w propylene glycol; about 0% w/w to about 2.7% w/w ethanol, and all values in between, e.g., about 1% w/w, about 2% w/w, and about 2.5% w/w; about 0.005% w/w to about 0.01% w/w of an antioxidant; about 0.008% w/w to about 0.12% w/w of a sweetener agent; about 0.008% w/w to about 0.08% w/w of a flavoring agent; and a vehicle comprising glycerin in an amount of about 79% w/w to about 96% w/w; wherein the amount of clopidogrel is based on the mass amount of clopidogrel per mass amount of solution.

Yet another aspect relates to an oral pharmaceutical solution comprising about 1.0% w/w to about 1.4% w/w clopidogrel or a pharmaceutically acceptable salt thereof, about 2.8% w/w to about 16.3% w/w propylene glycol; about 0% w/w to about 2.7% w/w ethanol; about 0.005% w/w to about 0.01% w/w of an antioxidant (e.g., butylated hydroxyanisole); about 0.008% w/w to about 0.12% w/w of a sweetener agent (e.g., sucralose); about 0.008% w/w to about 0.08% w/w of a flavoring agent; and a vehicle comprising glycerin in an amount of about 79% w/w to about 96% w/w; wherein the amount of clopidogrel is based on the mass amount of clopidogrel per mass amount of solution.

A further aspect relates to an oral pharmaceutical solution comprising about 1.5% w/w to about 1.8% w/w clopidogrel bisulfate; about 2.8% w/w to about 16% w/w propylene glycol; about 0% w/w to about 2.6% w/w ethanol; about 0.005% w/w to about 0.01% w/w of an antioxidant; about 0.008% w/w to about 0.12% w/w of a sweetener agent; about 0.008% w/w to about 0.08% w/w of a flavoring agent; and a vehicle comprising glycerin in an amount of about 79% w/w to about 96% w/w; wherein the amount of clopidogrel bisulfate is based on the mass amount of clopidogrel per mass amount of solution.

A further aspect relates to an oral pharmaceutical solution comprising about 1.5% w/w to about 1.8% w/w clopidogrel bisulfate; about 2.8% w/w to about 16% w/w propylene glycol; about 0% w/w to about 2.6% w/w ethanol; about 0.005% w/w to about 0.01% w/w of an antioxidant (e.g., butylated hydroxyanisole); about 0.008% w/w to about 0.12% w/w of a sweetener agent (e.g., sucralose); about 0.008% w/w to about 0.08% w/w of a flavoring agent; and a vehicle comprising glycerin in an amount of about 79% w/w to about 96% w/w; wherein the amount of clopidogrel bisulfate is based on the mass amount of clopidogrel per mass amount of solution.

### Third Aspect

Another aspect relates to an oral pharmaceutical solution comprising about 13 mg/mL to about 17 mg/mL clopidogrel or a pharmaceutically acceptable salt thereof; about 70 mg/mL to about 200 mg/mL propylene glycol; about 16 mg/mL to about 33 mg/mL ethanol; one or more pharmaceutically acceptable excipients; and a vehicle comprising glycerin; wherein the amount of clopidogrel is based on the mass amount of clopidogrel per milliliter of solution.

Another aspect relates to an oral pharmaceutical solution comprising about 1.0% w/w to about 1.4% w/w clopidogrel or a pharmaceutically acceptable salt thereof, and all values in between, e.g., about 1.2% w/w; about 5.6% w/w to about 16% w/w propylene glycol, and all values in between, e.g., about 6% w/w, about 7% w/w, about 8% w/w, about 9% w/w, about 10% w/w, about 11% w/w, about 12% w/w, about 13% w/w, about 14% w/w, and about 15% w/w; about 1.3% w/w to about 2.7% w/w ethanol, and all values in between, e.g., about 1.7% w/w, about 2.2% w/w, and about 2.5% w/w; one or more pharmaceutically acceptable excipients; and a vehicle comprising glycerin in an amount of about 79% w/w to about 91% w/w and all values in between, e.g., about 80% w/w, about 82% w/w, about 84% w/w, about 86% w/w, about 88% w/w, and about 90% w/w; wherein the amount of clopidogrel is based on the mass amount of clopidogrel per mass amount of solution.

Yet another aspect relates to an oral pharmaceutical solution comprising about 1.0% w/w to about 1.4% w/w clopidogrel or a pharmaceutically acceptable salt thereof, about 5.6% w/w to about 16% w/w propylene glycol; about 1.3% w/w to about 2.7% w/w ethanol; about 0.005% w/w to about 0.01% w/w of an antioxidant; about 0.008% w/w to about 0.12% w/w of a sweetener agent; about 0.008% w/w to about 0.08% w/w of a flavoring agent; and a vehicle comprising glycerin in an amount of about 79% w/w to about 91% w/w; wherein the amount of clopidogrel is based on the mass amount of clopidogrel per mass amount of solution.

Yet another aspect relates to an oral pharmaceutical solution comprising about 1.4% w/w to about 1.8% w/w clopidogrel or a pharmaceutically acceptable salt thereof, about 5.6% w/w to about 16% w/w propylene glycol; about 1.3% w/w to about 2.7% w/w ethanol; about 0.005% w/w to about 0.01% w/w of an antioxidant (e.g., butylated hydroxyanisole); about 0.008% w/w to about 0.12% w/w of a sweetener agent (e.g., sucralose); about 0.008% w/w to about 0.08% w/w of a flavoring agent; and a vehicle comprising glycerin in an amount of about 79% w/w to about 92% w/w; wherein the amount of clopidogrel is based on the mass amount of clopidogrel per mass amount of solution.

A further aspect relates to an oral pharmaceutical solution comprising about 1.4% w/w to about 1.8% w/w clopidogrel bisulfate; about 5.6% w/w to about 16% w/w propylene glycol; about 1.3% w/w to about 2.7% w/w ethanol; about 0.005% w/w to about 0.01% w/w of an antioxidant; about 0.008% w/w to about 0.12% w/w of a sweetener agent; about 0.008% w/w to about 0.08% w/w of a flavoring agent; and a vehicle comprising glycerin in an amount of about 79% w/w to about 92% w/w; wherein the amount of clopidogrel bisulfate is based on the mass amount of clopidogrel per mass amount of solution.

A further aspect relates to an oral pharmaceutical solution comprising about 1.4% w/w to about 1.8% w/w clopidogrel bisulfate; about 5.6% w/w to about 16% w/w propylene glycol; about 1.3% w/w to about 2.7% w/w ethanol; about 0.005% w/w to about 0.01% w/w of an antioxidant (e.g., butylated hydroxyanisole); about 0.008% w/w to about 0.12% w/w of a sweetener agent (e.g., sucralose); about 0.008% w/w to about 0.08% w/w of a flavoring agent; and a vehicle comprising glycerin in an amount of about 79% w/w to about 92% w/w; wherein the amount of clopidogrel bisulfate is based on the mass amount of clopidogrel per mass amount of solution.

### Fourth Aspect

Another aspect relates to an oral pharmaceutical solution comprising about 15 mg/mL clopidogrel or a pharmaceutically acceptable salt thereof, about 70 mg/mL to about 200 mg/mL propylene glycol; about 16 mg/mL to about 33 mg/mL ethanol; one or more pharmaceutically acceptable excipients; and a vehicle comprising glycerin; wherein the amount of clopidogrel is based on the mass amount of clopidogrel per milliliter of solution.

Another aspect relates to an oral pharmaceutical solution comprising about 1.2% w/w clopidogrel or a pharmaceutically acceptable salt thereof, about 5.6% w/w to about 16% w/w propylene glycol, and all values in between, e.g., about 6% w/w, about 7% w/w, about 8% w/w, about 9% w/w, about 10% w/w, about 11% w/w, about 12% w/w, about 13% w/w, about 14% w/w, and about 15% w/w; about 1.3% w/w to about 2.6% w/w ethanol, and all values in between, e.g., about 1.7% w/w, about 2.2% w/w, and about 2.5% w/w; one or more pharmaceutically acceptable excipients; and a vehicle comprising glycerin in an amount of about 80% w/w to about 92% w/w, and all values in between, e.g., about 82% w/w, about 84% w/w, about 86% w/w, about 88% w/w, and about 90% w/w; wherein the amount of clopidogrel is based on the mass amount of clopidogrel per mass amount of solution.

Yet another aspect relates to an oral pharmaceutical solution comprising about 1.2% w/w clopidogrel or a pharmaceutically acceptable salt thereof, about 5.6% w/w to about 16% w/w propylene glycol; about 1.3% w/w to about 2.7% w/w ethanol; about 0.005% w/w to about 0.01% w/w of an antioxidant; about 0.008% w/w to about 0.12% w/w of a sweetener agent; about 0.008% w/w to about 0.08% w/w of a flavoring agent; and a vehicle comprising glycerin in an amount of about 80% w/w to about 92% w/w; wherein the amount of clopidogrel is based on the mass amount of clopidogrel per mass amount of solution.

Yet another aspect relates to an oral pharmaceutical solution comprising about 1.2% w/w clopidogrel or a pharmaceutically acceptable salt thereof, about 5.6% w/w to about 16% w/w propylene glycol; about 1.3% w/w to about 2.7% w/w ethanol; about 0.005% w/w to about 0.01% w/w of an antioxidant (e.g., butylated hydroxyanisole); about 0.008% w/w to about 0.12% w/w of a sweetener agent (e.g., sucralose); about 0.008% w/w to about 0.08% w/w of a flavoring agent; and a vehicle comprising glycerin in an amount of about 80% w/w to about 92% w/w; wherein the amount of clopidogrel is based on the mass amount of clopidogrel per mass amount of solution.

A further aspect relates to an oral pharmaceutical solution comprising about 1.6% w/w clopidogrel bisulfate; about 5.6% w/w to about 16% w/w propylene glycol; about 1.3% w/w to about 2.7% w/w ethanol; about 0.005% w/w to about 0.01% w/w of an antioxidant; about 0.008% w/w to about 0.12% w/w of a sweetener agent; about 0.008% w/w to about 0.08% w/w of a flavoring agent; and a vehicle comprising glycerin in an amount of about 79% w/w to about 91% w/w; wherein the amount of clopidogrel bisulfate is based on the mass amount of clopidogrel per mass amount of solution.

A further aspect relates to an oral pharmaceutical solution comprising about 1.6% w/w clopidogrel bisulfate; about 5.6% w/w to about 16% w/w propylene glycol; about 1.3% w/w to about 2.7% w/w ethanol; about 0.005% w/w to about 0.01% w/w of an antioxidant (e.g., butylated hydroxyanisole); about 0.008% w/w to about 0.12% w/w of a sweetener agent (e.g., sucralose); about 0.008% w/w to about 0.08% w/w of a flavoring agent; and a vehicle comprising glycerin in an amount of about 79% w/w to about 91% w/w; wherein the amount of clopidogrel bisulfate is based on the mass amount of clopidogrel per mass amount of solution.

### Fifth Aspect

Another aspect relates to an oral pharmaceutical solution comprising about 15 mg/mL clopidogrel or a pharmaceutically acceptable salt thereof; about 70 mg/mL propylene glycol; from about 32 to about 33 mg/mL ethanol; one or more pharmaceutically acceptable excipients; and a vehicle comprising glycerin; wherein the ethanol content is about 4% v/v; and wherein the amount of clopidogrel is based on the mass amount of clopidogrel per milliliter of solution.

Another aspect relates to an oral pharmaceutical solution comprising about 1.2% w/w clopidogrel or a pharmaceutically acceptable salt thereof; about 5.6% w/w to 16% w/w propylene glycol, and all values in between, e.g., about 6% w/w, about 7% w/w, about 8% w/w, about 9% w/w, about 10% w/w, about 11% w/w, about 12% w/w, about 13% w/w, about 14% w/w, and about 15% w/w; about 2.6% w/w to about 2.7% w/w ethanol; one or more pharmaceutically acceptable excipients; and a vehicle comprising glycerin in an amount of about 80% w/w to about 90% w/w; wherein the amount of clopidogrel is based on the mass amount of clopidogrel per mass amount of solution.

Yet another aspect relates to an oral pharmaceutical solution comprising about 1.2% w/w clopidogrel or a pharmaceutically acceptable salt thereof, about 5.6% w/w to about 16% w/w propylene glycol; about 2.6% w/w to about 2.7% w/w ethanol; about 0.005% w/w to about 0.01% w/w of an antioxidant; about 0.008% w/w to about 0.12% w/w of a sweetener agent; about 0.008% w/w to about 0.08% w/w of a flavoring agent; and a vehicle comprising glycerin in an amount of about 80% w/w to about 90% w/w; wherein the amount of clopidogrel is based on the mass amount of clopidogrel per mass amount of solution.

Yet another aspect relates to an oral pharmaceutical solution comprising about 1.2% w/w clopidogrel or a pharmaceutically acceptable salt thereof; about 5.6% w/w to 16% w/w propylene glycol; about 2.6% w/w to about 2.7% w/w ethanol; about 0.005% w/w to about 0.01% w/w of an antioxidant (e.g., butylated hydroxyanisole); about 0.008% w/w to about 0.12% w/w of a sweetener agent (e.g., sucralose); about 0.008% w/w to about 0.08% w/w of a flavoring agent; and a vehicle comprising glycerin in an amount of about 80% w/w to about 90% w/w; wherein the amount of clopidogrel is based on the mass amount of clopidogrel per mass amount of solution.

A further aspect relates to an oral pharmaceutical solution comprising about 1.6% w/w clopidogrel bisulfate; about 5% w/w to 16% w/w propylene glycol; about 2.6% w/w to about 2.7% w/w ethanol; about 0.005% w/w to about 0.01% w/w of an antioxidant; about 0.008% w/w to about 0.12% w/w of a sweetener agent; about 0.008% w/w to about 0.08% w/w of a flavoring agent; and a vehicle comprising glycerin in an amount of about 79% w/w to about 90% w/w; wherein the amount of clopidogrel bisulfate is based on the mass amount of clopidogrel per mass amount of solution.

A further aspect relates to an oral pharmaceutical solution comprising about 1.6% w/w clopidogrel bisulfate; about 5% w/w to 16% w/w propylene glycol; about 2.6% w/w to about 2.7% w/w ethanol; about 0.005% w/w to about 0.01% w/w of an antioxidant (e.g., butylated hydroxyanisole); about 0.008% w/w to about 0.12% w/w of a sweetener agent (e.g., sucralose); about 0.008% w/w to about 0.08% w/w of a flavoring agent; and a vehicle comprising glycerin in an amount of about 79% w/w to about 90% w/w; wherein the amount of clopidogrel bisulfate is based on the mass amount of clopidogrel per mass amount of solution.

Sixth Aspect

Another aspect relates to an oral pharmaceutical solution comprising about 13 mg/mL to 17 mg/mL clopidogrel or a pharmaceutically acceptable salt thereof, about 70 mg/mL to about 200 mg/mL propylene glycol; about 0 mg/mL ethanol (viz., no added ethanol); one or more pharmaceutically acceptable excipients; and a vehicle comprising glycerin; wherein the ethanol content is not more than about 0% v/v (or about 0% w/w); and wherein the amount of clopidogrel is based on the mass amount of clopidogrel per milliliter of solution.

Another aspect relates to an oral pharmaceutical solution comprising about 1.0% w/w to about 1.4% w/w clopidogrel or a pharmaceutically acceptable salt thereof; about 5.6% w/w to about 16% w/w propylene glycol, and all values in between, e.g., about 6% w/w, about 7% w/w, about 8% w/w, about 9% w/w, about 10% w/w, about 11% w/w, about 12% w/w, about 13% w/w, about 14% w/w, and about 15% w/w; about 0% w/w ethanol; one or more pharmaceutically acceptable excipients; and a vehicle comprising glycerin in an amount of about 82% w/w to about 93% w/w; wherein the amount of clopidogrel is based on the mass amount of clopidogrel per mass amount of solution.

Yet another aspect relates to an oral pharmaceutical solution comprising about 1.0% w/w to about 1.4% w/w clopidogrel or a pharmaceutically acceptable salt thereof, about 5.6% w/w to about 16% w/w propylene glycol; about 0% w/w ethanol; about 0.005% w/w to about 0.01% w/w of an antioxidant; about 0% w/w to about 0.12% w/w of a sweetener agent; about 0.008% w/w to about 0.08% w/w of a flavoring agent; and a vehicle comprising glycerin in an amount of about 82% w/w to about 93% w/w; wherein the amount of clopidogrel is based on the mass amount of clopidogrel per mass amount of solution.

Yet another aspect relates to an oral pharmaceutical solution comprising about 1.0% w/w to about 1.4% w/w clopidogrel or a pharmaceutically acceptable salt thereof, about 5.6% w/w to about 16% w/w propylene glycol; about 0% w/w ethanol; about 0.005% w/w to about 0.01% w/w of an antioxidant (e.g., butylated hydroxyanisole); about 0% w/w to about 0.12% w/w of a sweetener agent (e.g., sucralose); about 0.008% w/w to about 0.08% w/w of a flavoring agent; and a vehicle comprising glycerin in an amount of about 82% w/w to about 93% w/w; wherein the amount of clopidogrel is based on the mass amount of clopidogrel per mass amount of solution.

A further aspect relates to an oral pharmaceutical solution comprising about 1.4% w/w to about 1.8% w/w clopidogrel bisulfate; about 5.6% w/w to about 16% w/w propylene glycol; about 0% w/w ethanol; about 0.005% w/w to about 0.011% w/w of an antioxidant; about 0% w/w to about 0.12% w/w of a sweetener agent; about 0.008% w/w to about 0.08% w/w of a flavoring agent; and a vehicle comprising glycerin in an amount of about 79% w/w to about 90% w/w; wherein the amount of clopidogrel bisulfate is based on the mass amount of clopidogrel per mass amount of solution.

A further aspect relates to an oral pharmaceutical solution comprising about 1.4% w/w to about 1.8% w/w clopidogrel bisulfate; about 5.6% w/w to about 16% w/w propylene glycol; about 0% w/w ethanol; about 0.005% w/w to about 0.01% w/w of an antioxidant (e.g., butylated hydroxyanisole); about 0% w/w to about 0.12% w/w of a sweetener agent (e.g., sucralose); about 0.008% w/w to about 0.08% w/w of a flavoring agent; and a vehicle comprising glycerin in an amount of about 79% w/w to about 90% w/w; wherein the amount of clopidogrel bisulfate is based on the mass amount of clopidogrel per mass amount of solution.

### Seventh Aspect

Another aspect relates to an oral pharmaceutical solution comprising about 15 mg/mL clopidogrel or a pharmaceutically acceptable salt thereof, about 70 mg/mL to about 200 mg/mL propylene glycol; about 0 mg/mL ethanol (viz., no added ethanol); one or more pharmaceutically acceptable excipients; and a vehicle comprising glycerin; wherein the ethanol content is not more than about 0% v/v (or about 0% w/w); and wherein the amount of clopidogrel is based on the mass amount of clopidogrel per milliliter of solution.

Another aspect relates to an oral pharmaceutical solution comprising about 1.2% w/w clopidogrel or a pharmaceutically acceptable salt thereof; about 5.6% w/w to 16% w/w propylene glycol; about 0% w/w ethanol; one or more pharmaceutically acceptable excipients; and a vehicle comprising glycerin in an amount of about 82% w/w to about 93% w/w; wherein the amount of clopidogrel is based on the mass amount of clopidogrel per mass amount of solution.

Yet another aspect relates to an oral pharmaceutical solution comprising about 1.2% w/w clopidogrel or a pharmaceutically acceptable salt thereof; about 5.6% w/w to 16% w/w propylene glycol; about 0% w/w ethanol; about 0.005% w/w to about 0.01% w/w of an antioxidant; about 0% w/w to about 0.12% w/w of a sweetener agent; about 0.008% w/w to about 0.08% w/w of a flavoring agent; and a vehicle comprising glycerin in an amount of about 82% w/w to about 93% w/w; wherein the amount of clopidogrel is based on the mass amount of clopidogrel per mass amount of solution.

Yet another aspect relates to an oral pharmaceutical solution comprising about 1.2% w/w clopidogrel or a pharmaceutically acceptable salt thereof; about 5.6% w/w to 16% w/w propylene glycol; about 0% w/w ethanol; about 0.005% w/w to about 0.01% w/w of an antioxidant (e.g., butylated hydroxyanisole); about 0% w/w to about 0.12% w/w of a sweetener agent (e.g., sucralose); about 0.008% w/w to about 0.08% w/w of a flavoring agent; and a vehicle comprising glycerin in an amount of about 82% w/w to about 93% w/w; wherein the amount of clopidogrel is based on the mass amount of clopidogrel per mass amount of solution.

A further aspect relates to an oral pharmaceutical solution comprising about 1.6% w/w clopidogrel bisulfate; about 5.6% w/w to 16% w/w propylene glycol; about 0% w/w ethanol; about 0.005% w/w to about 0.01% w/w of an antioxidant; about 0% w/w to about 0.12% w/w of a sweetener agent; about 0.008% w/w to about 0.08% w/w of a flavoring agent; and a vehicle comprising glycerin in an amount of about 82% w/w to about 92% w/w; wherein the amount of clopidogrel bisulfate is based on the mass amount of clopidogrel per mass amount of solution.

A further aspect relates to an oral pharmaceutical solution comprising about 1.6% w/w clopidogrel bisulfate; about 5.6% w/w to 16% w/w propylene glycol; about 0% w/w ethanol; about 0.005% w/w to about 0.01% w/w of an antioxidant (e.g., butylated hydroxyanisole); about 0% w/w to about 0.12% w/w of a sweetener agent (e.g., sucralose); about 0.008% w/w to about 0.08% w/w of a flavoring agent; and a vehicle comprising glycerin in an amount of about 82% w/w to about 92% w/w; wherein the amount of clopidogrel bisulfate is based on the mass amount of clopidogrel per mass amount of solution.

### Eighth Aspect

Another aspect relates to an oral pharmaceutical solution comprising about 13 mg/mL to 17 mg/mL clopidogrel or a pharmaceutically acceptable salt thereof, about 0 mg/mL propylene glycol (viz., no added propylene glycol); about 0 mg/mL ethanol (viz., no added ethanol); one or more pharmaceutically acceptable excipients; and a vehicle comprising glycerin; wherein the amount of clopidogrel is based on the mass amount of clopidogrel per milliliter of solution.

Another aspect relates to an oral pharmaceutical solution comprising about 1.1% w/w to about 1.4% w/w clopidogrel or a pharmaceutically acceptable salt thereof; one or more pharmaceutically acceptable excipients; and a vehicle comprising glycerin in an amount of about 98% w/w to about 99% w/w; wherein the amount of clopidogrel is based on the mass amount of clopidogrel per mass amount of solution.

Yet another aspect relates to an oral pharmaceutical solution comprising about 1.1% w/w to about 1.4% w/w clopidogrel or a pharmaceutically acceptable salt thereof; about 0.005% w/w to about 0.01% w/w of an antioxidant; about 0% w/w to about 0.12% w/w of a sweetener agent; about 0.008% w/w to about 0.08% w/w of a flavoring agent; and a vehicle comprising glycerin in an amount of about 98% w/w to about 99% w/w; wherein the amount of clopidogrel is based on the mass amount of clopidogrel per mass amount of solution.

Yet another aspect relates to an oral pharmaceutical solution comprising about 1.1% w/w to about 1.4% w/w clopidogrel or a pharmaceutically acceptable salt thereof; about 0.005% w/w to about 0.01% w/w of an antioxidant (e.g., butylated hydroxyanisole); about 0% w/w to about 0.12% w/w of a sweetener agent (e.g., sucralose); about 0.008% w/w to about 0.08% w/w of a flavoring agent; and a vehicle comprising glycerin in an amount of about 98% w/w to about 99% w/w; wherein the amount of clopidogrel is based on the mass amount of clopidogrel per mass amount of solution.

A further aspect relates to an oral pharmaceutical solution comprising about 1.4% w/w to about 1.8% w/w clopidogrel bisulfate; about 0.005% w/w to about 0.01% w/w of an antioxidant; about 0% w/w to about 0.12% w/w of a sweetener agent; about 0.008% w/w to about 0.08% w/w of a flavoring agent; and a vehicle comprising glycerin in an amount of about 98% w/w to about 99% w/w; wherein the amount of clopidogrel bisulfate is based on the mass amount of clopidogrel per mass amount of solution.

A further aspect relates to an oral pharmaceutical solution comprising about 1.4% w/w to about 1.8% w/w clopidogrel bisulfate; about 0.005% w/w to about 0.01% w/w of an antioxidant (e.g., butylated hydroxyanisole); about 0% w/w to about 0.12% w/w of a sweetener agent (e.g., sucralose); about 0.008% w/w to about 0.08% w/w of a flavoring agent; and a vehicle comprising glycerin in an amount of about 98% w/w to about 99% w/w; wherein the amount of clopidogrel bisulfate is based on the mass amount of clopidogrel per mass amount of solution.

### Ninth Aspect

Another aspect relates to an oral pharmaceutical solution comprising about 15 mg/mL clopidogrel or a pharmaceutically acceptable salt thereof, about 0 mg/mL propylene glycol (viz., no added propylene glycol); about 0 mg/mL ethanol (viz., no added ethanol); one or more pharmaceutically acceptable excipients; and a vehicle comprising glycerin; wherein the ethanol content is not more than about 0% v/v (or about 0% w/w to about 2.6% w/w); wherein the amount of clopidogrel is based on the mass amount of clopidogrel per milliliter of solution.

Another aspect relates to an oral pharmaceutical solution comprising about 1.2% w/w clopidogrel or a pharmaceutically acceptable salt thereof, one or more pharmaceutically acceptable excipients; and a vehicle comprising glycerin in an amount of about 98% w/w to about 99% w/w; wherein the amount of clopidogrel is based on the mass amount of clopidogrel per mass amount of solution.

Yet another aspect relates to an oral pharmaceutical solution comprising about 1.2% w/w clopidogrel or a pharmaceutically acceptable salt thereof; about 0.005% w/w to about 0.01% w/w of an antioxidant; about 0% w/w to about 0.12% w/w of a sweetener agent; about 0.008% w/w to about 0.08% w/w of a flavoring agent; and a vehicle comprising glycerin in an amount of about 98% w/w to about 99% w/w; wherein the amount of clopidogrel is based on the mass amount of clopidogrel per mass amount of solution.

Yet another aspect relates to an oral pharmaceutical solution comprising about 1.2% w/w clopidogrel or a pharmaceutically acceptable salt thereof; about 0.005% w/w to about 0.011% w/w of an antioxidant (e.g., butylated hydroxyanisole); about 0% w/w to about 0.12% w/w of a sweetener agent (e.g., sucralose); about 0.008% w/w to about 0.08% w/w of a flavoring agent; and a vehicle comprising glycerin in an amount of about 98% w/w to about 99% w/w; wherein the amount of clopidogrel is based on the mass amount of clopidogrel per mass amount of solution.

A further aspect relates to an oral pharmaceutical solution comprising about 1.6% w/w clopidogrel bisulfate; about 0.005% w/w to about 0.01% w/w of an antioxidant; about 0% w/w to about 0.12% w/w of a sweetener agent; about 0.008% w/w to about 0.08% w/w of a flavoring agent; and a vehicle comprising glycerin in an amount of about 98% w/w to about 99% w/w; wherein the amount of clopidogrel bisulfate is based on the mass amount of clopidogrel per mass amount of solution.

A further aspect relates to an oral pharmaceutical solution comprising about 1.6% w/w clopidogrel bisulfate; about 0.005% w/w to about 0.01% w/w of an antioxidant (e.g., butylated hydroxyanisole); about 0% w/w to about 0.12% w/w of a sweetener agent (e.g., sucralose); about 0.008% w/w to about 0.08% w/w of a flavoring agent; and a vehicle comprising glycerin in an amount of about 98% w/w to about 99% w/w; wherein the amount of clopidogrel bisulfate is based on the mass amount of clopidogrel per mass amount of solution.

### Additional Aspects

The liquid solution may comprise further excipients than those listed above including, for example, a preservative. Other known pharmaceutical excipients may be used in the ordinary amounts for their normal purposes, so long as they do not negatively affect the effectiveness or stability of the solution.

Preservatives may also be included to prevent the growth of microorganisms during the product manufacturing and shelf life. Preservatives may be selected from but not limited to benzoic acid, sorbic acid, butyl paraben, propyl paraben, and methyl paraben, or a pharmaceutically acceptable salt thereof (e.g., sodium benzoate, potassium sorbate, and the like). In an alternative aspect, the oral pharmaceutical solution may comprise methyl paraben, propyl paraben, a salt thereof, or a combination thereof.

Based on unpredictability of developing a liquid solution of clopidogrel capable of retaining physicochemical stability, only a few formulations had sufficient physicochemical shelf stability (e.g., 24 months), acceptable bioavailability (e.g., equivalent to the approved solid dose formulation), and contained levels of excipients that were deemed safe for human exposure.

The oral pharmaceutical solution disclosed herein does not contain a surfactant/solubilizing agent (e.g., polyethylene glycol-15-hydroxystearate (SOLUTOL®), polysorbate 80 (TWEEN 80®), or polyethylene glycol 660 12-hydroxystearate (SOLUTOL HS 15®)). Further, the oral pharmaceutical solution disclosed herein does not contain a cyclodextrin (e.g., a sulfoalkyl ether cyclodextrin or hydroxypropyl-β-cyclodextrin)). One may appreciate that the expression “comprising” may be replaced with the expression “consisting of” or “consist of” to reflect a formulation disclosed herein.

### Dosage Dispensing Unit and Stability

Some aspects relate to a dosage dispensing unit that contains an oral pharmaceutical solution disclosed herein. In some aspects, a desired amount of the oral pharmaceutical solution described herein is withdrawn from a dosage dispensing unit, and administered to a patient. In some aspects, the oral pharmaceutical solutions described herein is withdrawn from the dosage dispensing unit by use of a direct dosing device. Thus, in some aspects, the present invention also relates to a direct dosing device that contains the oral pharmaceutical solution described herein.

The oral pharmaceutical solutions described herein may be stored in a pharmaceutically acceptable container-closure system, such as, for example, a clear glass bottle, an amber glass bottle, an HDPE bottle, an amber PET bottle, a clear PET bottle, an amber HDPE bottle, a cup, a syringe, etc. The bottle volume is, for example, about 150 mL to about 200 mL, e.g., 185 mL, with a formulation volume of about 150 mL. A photostability study (viz., total light exposure of 1.2 million lux hours and an integrated near ultraviolet energy of not less than 200-watt hours/square meter) suggests an increased amount of Impurity A upon exposure in a clear bottle, while amber colored bottles showed no comparable increase in Impurity A. An amber colored bottle has a light transmission of, for example, less than 5%, less than 4%, less than 3%, less than 2%, or less than 1% at any wavelength between 290 nm and 450 nm. Accordingly, an oral pharmaceutical solution described herein may be stored in a pharmaceutically acceptable container-closure system, such as, for example, an amber glass bottle, an HDPE bottle, an amber PET bottle, or an amber HDPE bottle.

One aspect relates to a bottled product comprising a formulation described herein. In one aspect, the bottled product comprises an amber glass bottle (see DMF No. 14003) with a pharmaceutically acceptable closure (such as a 28 mm Polypropylene TE-CRC cap with EPE liner, see DMF No. 18371).

Oral pharmaceutical solutions disclosed herein results in a clopidogrel stability heretofore not observed.

An oral pharmaceutical solution disclosed herein has a clopidogrel content of 100±10% clopidogrel labeled content when stored for about W-months at 25±2° C. and 60±5% relative humidity, where W is 1, 3, 6, 9, 12, 18, 24, 30, 36, 42, or 48.

Additionally, an oral pharmaceutical solution disclosed herein has an amount of Impurity A of not more than about 1.2% w/w when stored for about X-months at 25±2° C. and 60±5% relative humidity, where X is 1, 3, 6, 9, 12, 18, 24, 30, 36, 42, or 48.

Moreover, an oral pharmaceutical solution contemplated herein has an amount of Impurity C of not more than about 1.5% w/w when stored for at least about Y-months at 25±2° C. and 60±5% relative humidity, where Y is 1, 3, 6, 9, 12, 18, 24, 30, 36, 42, or 48.

Further, an oral pharmaceutical solution contemplated herein has an amount of Impurity F of not more than about 0.2% w/w when stored for at least about Z-months at 25±2° C. and 60±5% relative humidity, where Z is 1, 3, 6, 9, 12, 18, 24, 30, 36, 42, or 48.

An oral pharmaceutical solution contemplated herein has a density of about 1.22 to about 1.27 g/mL when stored for at least about Z-months at 25±2° C. and 60±5% relative humidity, where Z is 1, 3, 6, 9, 12, 18, 24, 30, 36, 42, or 48.

### Methods of Use

An oral pharmaceutical solution disclosed herein may be used in a manner consistent with the PLAVIX® PI. For instance, PLAVIX® (clopidogrel bisulfate), in combination with aspirin, is indicated to reduce the rate of myocardial infarction (MI) and stroke in patients with non-ST-segment elevation ACS (unstable angina [UA]/non-ST-elevation myocardial infarction [NSTEMI]), including patients who are to be managed medically and those who are to be managed with coronary revascularization.

Additionally, PLAVIX® (clopidogrel bisulfate), in combination with aspirin, is indicated to reduce the rate of myocardial infarction and stroke in patients with acute ST-elevation myocardial infarction (STEMI) who are to be managed medically.

Further, in patients with established peripheral arterial disease or with a history of recent myocardial infarction (MI) or recent stroke PLAVIX® (clopidogrel bisulfate), is indicated to reduce the rate of MI and stroke.

Accordingly, contemplated herein is a method for the treatment of a condition, which comprises administering to a patient in need thereof a therapeutically effective amount of a oral pharmaceutical solution disclosed herein, optionally, in combination with aspirin, wherein the condition comprises acute coronary syndrome, myocardial infarction, stroke, peripheral arterial disease, or a combination thereof.

The term “therapeutically effective amount” or effective dose” as used herein refers to the amount or dose of clopidogrel that is sufficient to initiate therapeutic response in a patient.

Additionally, contemplated herein is a method for treating a condition, which comprises administering to a patient in need thereof, with or without aspirin, a therapeutically effective amount of an oral pharmaceutical solution disclosed herein consistent with any one of the conditions identified in the PLAVIX® PI, wherein the condition is selected from (i) reduce the rate of myocardial infarction (MI) and stroke in patients with non-ST-segment elevation ACS (unstable angina [UA]/non-ST-elevation myocardial infarction [NSTEMI]), including patients who are to be managed medically and those who are to be managed with coronary revascularization, (ii) reduce the rate of myocardial infarction and stroke in patients with acute ST-elevation myocardial infarction (STEMI) who are to be managed medically, and (iii) reduce the rate of MI and stroke.

One will appreciate that an antiplatelet effect may be necessary in certain patients by administering a single loading dose (e.g., 300-mg), which may then be followed by a daily maintenance dose (e.g., 75-mg). An oral pharmaceutical solution disclosed herein is advantageous because the loading and maintenance doses do not require different dosage forms.

Aspects of the oral pharmaceutical solution described herein may be understood with reference to the development work and exemplified embodiments, which are not meant to limit the subject matter claimed herein.

### Development Work

Amounts of clopidogrel and other components identified herein were determined chromatographically, e.g., HPLC. USP 28 at 516-517. Antimicrobial Effectiveness Testing studies were conducted according to USP Chapter <51>.

### Effect of Antioxidant on Presence of Impurities

Experiments were carried out to evaluate the effect of antioxidant on the stability of clopidogrel. Two batches were prepared: (i) Composition 1 comprises 0.1 mg/mL BHA as the antioxidant, and (ii) Composition 2 comprises 0.1 mg/mL BHT as the antioxidant. The compositions are presented in Table 1, and were prepared according to the procedures detailed below.

TABLE 1
Clopidogrel Compositions 15 mg/mL with different antioxidants

| | Composition ID | | | |
|---|---|---|---|---|
| | Composition 1 | | Composition 2 | |
| | Quantity | Quantity | Quantity | Quantity |
| Ingredients | (mg)/mL | (gm)/batch | (mg)/mL | (gm)/batch |
| Clopidogrel | 19.58 ≈ | 19.58 | 19.58 ≈ | 19.58 |
| hydrogen | 15.0 | | 15.0 | |
| sulphate eq. | | | | |
| to Clopidogrel | | | | |
| Propylene | 70.0 | 70.0 | 70.0 | 70.0 |
| glycol | | | | |
| Ethanol | 0.04 mL ≈ | 31.6 | 0.04 mL ≈ | 31.6 |
| | 31.6 | | 31.6 | |
| Butylated | 0.10 | 0.10 | — | — |
| hydroxyanisole | | | | |
| (BHA) | | | | |
| Butylated | — | — | 0.10 | 0.10 |
| hydroxytoluene | | | | |
| (BHT) | | | | |
| Sucralose | 1.0 | 1.0 | 1.0 | 1.0 |
| Frozen | 0.2 | 0.2 | 0.2 | 0.2 |
| Peppermint | | | | |
| Flavor | | | | |
| Glycerin | qs. to | qs. to | qs. to | qs. to |
| | 1 mL ≈ | 1 L ≈ | 1 mL ≈ | 1 L ≈ |
| | 1.245 gm | 1.245 gm | 1.245 gm | 1.245 gm |

### Batch Manufacturing Procedure for Composition 1:

1. Tare weight of SS manufacturing vessel S1: Add 1.0 kg of glycerin to the SS manufacturing vessel S1.
2. Tare weight of SS manufacturing vessel S2: Add 32 g of Ethanol from total batch size in SS manufacturing vessel S2.
3. Add dispensed quantity of butylated hydroxyanisole in manufacturing vessel S2 step 2, stir well until clear solution is obtained.
4. Add dispensed quantity sucralose of in manufacturing vessel S2 step 3, stir well until clear solution is obtained.
5. Add dispensed quantity propylene glycol of in manufacturing vessel S2 step 4, stir well until clear solution is obtained.
6. Add dispensed quantity of clopidogrel hydrogen sulphate in manufacturing vessel S2 step 5, stir well until clear solution is obtained.
7. Transfer the content of SS manufacturing vessel S2 of step 5, to vessel S1 of step 1. Stir well until clear homogeneous solution is obtained.
8. Add dispensed quantity of peppermint flavor in manufacturing vessel S1 step 7, stir well until clear solution is obtained.
9. Add remaining quantity of glycerin to make up the volume 1 L=1.245 kg and stirred by overhead stirrer in SS manufacturing vessel S1.

### Batch Manufacturing Procedure for Composition 2:

1. Tare weight of SS manufacturing vessel S1: Add 1.0 kg of Glycerin SS manufacturing vessel S1.
2. Tare weight of SS manufacturing vessel S2: Add 32 g of Ethanol from total batch size in SS manufacturing vessel S2.
3. Add dispensed quantity of butylated hydroxytoluene in manufacturing vessel S2 step 2, stir well until clear solution is obtained.
4. Add dispensed quantity Sucralose of in manufacturing vessel S2 step 3, stir well until clear solution is obtained.
5. Add dispensed quantity propylene glycol of in manufacturing vessel S2 step 4, stir well until clear solution is obtained.
6. Add dispensed quantity of Clopidogrel hydrogen sulphate in manufacturing vessel S2 step 5, stir well until clear solution is obtained.
7. Transfer the content of SS manufacturing vessel S2 of step 5, to vessel S1 of step 1. Stir well until clear homogeneous solution is obtained.
8. Add dispensed quantity of peppermint flavor in manufacturing vessel S1 step 7, stir well until clear solution is obtained.
9. Add remaining quantity of Glycerin to make up the volume 1 L=1.245 kg and stirred by overhead stirrer in SS manufacturing vessel S1.

Each of Compositions 1 and 2 were analyzed according to the parameters shown in Table 2 below at an initial time point (t=zero) immediately following preparation of the compositions, and after three months (t=3M) under storage at 40±2° C./75±5% RH.

TABLE 2
Stability Data for Compositions 1 and 2 after storage
at 40 ± 2° C./75 ± 5% RH

| | Specifi- | Composition 1 | | Composition 2 | |
|---|---|---|---|---|---|
| Parameters | cations | Initial | 3 M | Initial | 3 M |
| Assay of | 90.0%- | 95.6% | 95.4% | 95.5% | 94.0% |
| Clopidogrel | 110.0% LC | | | | |
| Content | NLT 60% LC | 94.3% | 88.4% | — | — |
| of BHA | | | | | |
| Content | NLT 60% LC | — | — | 45.5% | 40.6% |
| of BHT | | | | | |
| Imp. A | NMT 1.2% | 0.19% | 0.47% | 0.20% | 0.50% |
| Imp. C | NMT 1.5% | 0.63% | 1.50% | 0.71% | 2.23% |
| Imp. F | NMT 0.2% | 0.09% | 0.12% | 0.09% | 0.13% |
| SMUI | NMT 0.2% | 0.09% | 0.23% | 0.09% | 0.25% |
| TI | NMT 2.0% | 0.46% | 1.09% | 0.47% | 1.30% |

Abbreviations: LC (labelled content), NLT (not less than), NMT (not more than), SMUI (Single Maximum Unspecified Impurity), and TI (total impurities without Impurity C).

Composition 1 comprising BHA performed better with respect to the amount of impurities after subjected to accelerated conditions. Specifically, Composition 1 had only 1.5% w/w of Impurity C present in the final mixture, while Composition 2 had 2.23% w/w of Impurity C. Composition 2 containing BHT therefore saw about 50% higher Impurity C than the BHA counterpart. Moreover, the total impurities (without Impurity C) within Composition 1 were reduced compared to the BHT counterpart, again representing an improvement.

It is also noted within Table 2 that the preparation of Composition 2 resulted in a large decrease in BHT (45.5%) compared to that of BHA in Composition 1 (94.3%). Thus, BHA demonstrated an improved stability during the preparation and accelerated degradation testing compared to BHT.

One more formulation trial was taken wherein BHA was used in an amount of about 0.2 mg/mL. However, looking at the stability study results (not included herein), it was observed that BHA in an amount of about 0.1 mg/mL and BHA in an amount of about 0.2 mg/mL equally provided stability to the clopidogrel oral solution. Therefore, 0.1 mg/mL BHA was selected for further development.

### Effect of Ethanol on Stability During Thermal Cycling

Experiments were conducted to examine the effect of ethanol on the stability of liquid solutions of clopidogrel on exposure to by thermal cycling. Compositions 3-5 were subjected to a temperature cycle of −20±5° C. for 2 days followed by 40° C./75% RH for 2 days.

TABLE 3
Compositions of Clopidogrel, 15 mg/mL,
with different concentration of ethanol

| | Composition ID | | |
|---|---|---|---|
| | Composition 3 | Composition 4 | Composition 5 |
| | (4 L) | (1 L) | (1 L) |
| | Quantity | Quantity | Quantity |
| Ingredients | (mg)/mL | (mg)/mL | (mg)/mL |
| Clopidogrel hydrogen | 19.58 ≈ | 19.58 ≈ | 19.58 ≈ |
| sulphate eq. to | 15.0 | 15.0 | 15.0 |
| Clopidogrel | | | |
| Propylene glycol | 70.0 | 70.0 | 70.0 |
| Ethanol | 0.04 mL ≈ | 0.02 mL ≈ | 0.03 mL ≈ |
| | 31.6 (4%) | 15.8 (2%) | 23.7 (3%) |
| Butylated | 0.10 | 0.10 | 0.10 |
| hydroxyanisole | | | |
| Sucralose | 1.0 | 1.0 | 1.0 |
| Frozen Peppermint | 0.5 | 0.5 | 0.5 |
| Flavor | | | |
| Glycerin | qs. to 1 mL ≈ | qs. to 1 mL ≈ | qs. to 1 mL ≈ |
| | 1.245 gm | 1.245 gm | 1.245 gm |

### Batch Manufacturing Procedure for Compositions 3-5:

1. Tare weight of SS manufacturing vessel S1: Add dispensed quantity of glycerin in SS manufacturing vessel S1.
2. Tare weight of SS manufacturing vessel S2: Add dispensed quantity of ethanol from total batch size in SS manufacturing vessel S2.
3. Add dispensed quantity of butylated hydroxyanisole in manufacturing vessel S2 step 2, stir well until clear solution is obtained.
4. Add dispensed quantity sucralose of in manufacturing vessel S2 step 3, stir well until clear solution is obtained.
5. Add dispensed quantity propylene glycol of in manufacturing vessel S2 step 4, stir well until clear solution is obtained.
6. Add dispensed quantity of clopidogrel bisulfate in manufacturing vessel S2 step 5, stir well until clear solution is obtained.
7. Transfer the content of SS manufacturing vessel S2 of step 5, to vessel S1 of step 1. Stir well until clear homogeneous solution is obtained.
8. Add dispensed quantity of peppermint flavor in manufacturing vessel S1 step 7, stir well until clear solution is obtained.
9. Add remaining quantity of glycerin to make up the volume 1 L=1.245 kg and stirred by overhead stirrer in SS manufacturing vessel S1.

Each of Compositions 3-5 was clear upon compounding as noted by the processes above. For Composition 3 (4% v/v volume of ethanol), clopidogrel dissolved completely both at the end of batch and throughout thermal cycling. In Composition 4 (2% v/v ethanol), precipitation was observed when the sample was exposed to the thermal cycling study −20° C. for 2 days. In Composition 5 (3% v/v of ethanol), a hazy solution was observed when the sample was exposed to thermal cycling study −20° C. for 2 days.

Thus, evaluating the accelerated stability data of Composition 3, as well as the results of the thermal cycling study, it may be concluded that Composition 3 can withstand limited temperature excursions during the transportation process.

Despite the apparent benefit afforded by including ethanol in the solutions disclosed herein, at least with respect to solution stability, solutions of clopidogrel are also disclosed herein that are substantially ethanol free, replaced with, for example, propylene glycol, or glycerin or a combination thereof. Such oral pharmaceutical solutions of clopidogrel may be beneficially applied for pediatric formulations where any appreciable amount of ethanol is inappropriate. See, e.g., Weathermon at 51.

### Effect of Propylene Glycol on Clopidogrel Solubility

As stated above, a reduced ethanol content (e.g., Composition 4 (2% v/v ethanol)) may result in precipitation during a thermal cycle study. As an ethanol-free composition may be useful for pediatric patients (especially in view of the coadministration of aspirin), ethanol-free compositions were manufactured with varying amounts of propylene glycol. Table 4 shows the compositional makeup of three ethanol-free compositions with varying amounts of propylene glycol and glycerin.

TABLE 4
Compositions of Clopidogrel, 15 mg/mL,
with different concentration of PG

| | Composition 6 | Composition 7 | Composition 8^{‡} |
|---|---|---|---|
| Ingredients | (mg/mL) | (mg/mL) | (mg/mL) |
| Clopidogrel | 19.6 | 19.6 | 19.6 |
| bisulfate^{†} | | | |
| Propylene Glycol | 30 | 70 | 200 |
| Ethanol | — | — | — |
| Glycerin | Qs. to | Qs. to | Qs. to |
| | 1 mL^{§} | 1 mL^{§} | 1 mL^{§} |
| Batch Size (L) | 0.5 | 0.3 | 1.0 |
| Observations | Suspension | Solution | Solution |

^{†}19.6 mg/mL clopidogrel bisulfate is about 15 mg/mL clopidogrel.
^{‡}Composition 8 also included BHA (0.1 mg/mL) and raspberry flavor (1 mg/mL).
^{§}Target density of 1.245 g/mL.

### Batch Manufacturing Procedure for Compositions 6-8

1. Tare weight of SS manufacturing vessel S1: Add dispensed quantity of propylene glycol in SS manufacturing vessel S1.
2. For composition 8, add dispensed quantity of butylated hydroxyanisole in manufacturing vessel S1 step 1.
3. Add dispensed quantity of Clopidogrel bisulfate in manufacturing vessel S1 step 2.
4. Add dispensed quantity of glycerin in manufacturing vessel S1 step 3, stir well till clear solution is obtained.
5. For Composition 8, add dispensed quantity of raspberry flavor in manufacturing vessel S1 step 4, stir well till clear solution is obtained.
6. Add remaining quantity of glycerin to make up the volume (Composition 8 (300 mL=373.5 g) and Composition 8 (1 L=1.245 kg) and stirred by overhead stirrer in SS manufacturing vessel S1.

For Composition 6, a hazy solution (viz., a suspension) was observed, while Compositions 7-8 resulted in solutions (i.e., no particulate API). Based on these results, it was determined that a propylene glycol content of 30 mg/mL (or lower) resulted in a composition that included suspended clopidogrel bisulfate.

As related to stability, samples of Compositions 7-8 were analyzed by HPLC after storage at 40° C./75% RH. Composition 7 (70 mg/mL propylene glycol) had a lower Impurity A content after 3-months compared to Composition 8 (200 mg/mL propylene glycol).

These development studies showed that a propylene glycol content of from about 35 mg/mL to about 200 mg/mL provides for an ethanol-free, clopidogrel-containing solution in terms of dissolution and stability.

### Effect of Propylene Glycol on Stability

Experiments on the liquid solutions to evaluate the effect of propylene glycol on solution stability were conducted. Two solutions with different concentration of propylene glycol were evaluated and are presented below as Table 5 and prepared according to the manufacturing processes described below.

TABLE 5
Clopidogrel Oral Solution, 15 mg/mL, with
varied propylene glycol concentration

| | Formulation ID | |
|---|---|---|
| | Solution 1 | Solution 2 |
| Ingredients | Quantity (mg/mL) | Quantity (mg/mL) |
| Clopidogrel bisulfate | 19.58 (≈15) | 19.58 (≈15) |
| (clopidogrel eq.) | | |
| Propylene Glycol | 200.0 | 70.0 |
| Ethanol | — | 0.04 mL ≈ 31.6 mg/mL |
| Butylated Hydroxyanisole | 0.10 | 0.10 |
| Sucralose | — | 1.0 |
| Frozen Peppermint Flavor | — | 0.5 |
| Raspberry Flavor | 0.5 | — |
| Glycerin | qs. to 1 mL ≈ | qs. to 1 mL ≈ |
| | 1.245 g^{†} | 1.245 g^{†} |

^{†}Target density of 1.245 g/mL.

Batch Manufacturing Procedure for Solution 1 (Cf. Composition 8, Supra):
1. Tare weight of SS manufacturing vessel S1: Add dispensed quantity of Propylene glycol in SS manufacturing vessel S1.
2. Add dispensed quantity of butylated hydroxyanisole in manufacturing vessel S1 step 1.
3. Add dispensed quantity of clopidogrel bisulfate in manufacturing vessel S1 step 2.
4. Add dispensed quantity of glycerin in manufacturing vessel S1 step 3, stir well until clear solution is obtained.
5. Add dispensed quantity of raspberry flavor in manufacturing vessel S1 step 4, stir well until clear solution is obtained.
6. Add remaining quantity of glycerin to make up the volume 1 L=1.245 kg and stirred by overhead stirrer in SS manufacturing vessel S1.

### Batch Manufacturing Procedure for Solution 2:

1. Tare weight of SS manufacturing vessel S1: Add dispensed quantity of propylene glycol in SS manufacturing vessel S1.
2. Add dispensed quantity of ethanol in manufacturing vessel S1 step 1, stir well until clear solution is obtained.
3. Add dispensed quantity of butylated hydroxyanisole in manufacturing vessel S1 step 2, stir well until clear solution is obtained.
4. Add dispensed quantity of sucralose in manufacturing vessel S1 step 3, stir well until clear solution is obtained.
5. Add dispensed quantity of clopidogrel hydrogen sulphate in manufacturing vessel S1 step 4, stir well until clear solution is obtained.
6. Add dispensed quantity of propylene glycol in manufacturing vessel S1 step 5, stir well until clear solution is obtained.
7. Add dispensed quantity of glycerin in manufacturing vessel S1 step 6, stir well until clear solution is obtained.
8. Add remaining quantity of glycerin to make up the volume 1 L=1.245 kg and stirred by overhead stirrer in SS manufacturing vessel S1.

Solutions 1-2 prepared as above yielded a clear solution. Each of solutions 1-2 were analysed at an initial period immediately following preparation, and again after being subjected to accelerated degradation conditions. Each solution demonstrated stability of clopidogrel which remained constant for the duration of the stability test.

TABLE 6
Stability Data of Solutions 1-2 after storage at 40°
C. ± 2° C./75 ± 5% RH

| | Specifi- | Solution 1 | | Solution 2 | |
|---|---|---|---|---|---|
| Parameters | cations | Initial | 3 M | Initial | 3 M |
| Assay of | 90.0%- | 97.7% | 97.4% | 94.0% | 94.6% |
| Clopidogrel | 110.0% LC | | | | |
| Imp. A | NMT 1.2% | 0.32% | 0.63% | 0.06% | 0.44% |
| Imp. C | NMT 1.5% | NP | NP | 0.09% | 0.96% |
| SMUI | NMT 0.2% | 0.25% | 0.42% | 0.09% | 0.18% |
| TI | NMT 2.0% | 0.93% | 1.7% | 0.33% | 1.09% |

Abbreviations: LC (labelled content), NMT (not more than), SMUI (Single Maximum Unspecified Impurity), and TI (total impurities without Impurity C).

As per the Table 6, Solution 1 with 200 mg/mL propylene glycol (“PG”) showed increased degradation to Impurity A as compared to Solution 2 containing 70 mg/mL of PG. Specifically, Impurity A in Solution 1 was found at a level of 0.63%, when stored at 40±2° C./75%±5% RH for 3 months. Whereas Impurity-A in Solution 2 was found 0.44% at 40±2° C./75%±5% RH after 3 months. Based on above results, propylene glycol 70 mg/mL was finalized for further trial.

Solution 3—Clopidogrel, 15 mg/mL

Three batches were manufactured with the compositional makeup shown in Table 7.

TABLE 7
Solution of Clopidogrel, 15 mg/mL, batch size of 200 L

| | Formulation ID |
|---|---|
| | Solution 3 |
| Ingredients | Quantity (mg/mL) |
| Clopidogrel bisulfate | 19.58 (≈15) |
| (clopidogrel eq.) | |
| Propylene Glycol | 70.0 |
| Ethanol | 0.042 mL (≈33.18 mg/mL)^{‡} |
| Butylated Hydroxyanisole | 0.10 |
| Sucralose | 1.0 |
| Frozen Peppermint Flavor | 0.5 |
| Raspberry Flavor | — |
| Glycerin | qs. to 1 mL (≈1.230 g)^{†} |

^{‡}5% overage of ethanol providing ethanol content of 4.2% v/v (or about 4% v/v).
^{†}Target density of 1.230 g/mL with density specification of 1.205-1.255 g/mL.

Solution 3 was manufactured using process as described for Solution 2 (supra). Solution 3 was filtered and filled into 150 mL amber glass bottle, capped with 28 mm PP, EPE Wadded, child resistant closure, tamper evident (TE) ring.

Bottled solutions (Solution 3) were evaluated for stability at 25° C.±2° C./60±5% RH for 24 months in upright and inverted positions in a 150 mL amber glass bottle, capped with 28 mm PP, EPE Wadded, child resistant closure, tamper evident (TE) ring. Table 8 summarizes the observed data for the upright and inverted bottles.

TABLE 8
Stability Data of Upright (U) and Inverted (I) Bottles of Solution 3 after storage at 25° C. ± 2° C./60 ± 5% RH.

| | | Initial | | 3 M | | 6 M | | 9 M | | 12 M | | 18 M | | 24 M | |
|---|---|---|---|---|---|---|---|---|---|---|---|---|---|---|---|
| Parameters | Specification | U | I | U | I | U | I | U | I | U | I | U | I | U | I |
| Clopidogrel | 90.0%- | 99.0 | 99.0 | 98.4 | 99.3 | 98.8 | 99.6 | 99.8 | 99.5 | 99.4 | 99.1 | 99.9 | 99.1 | 95.5 | 96.5 |
| Assay | 110.0% LC | | | | | | | | | | | | | | |
| Content of | NLT 60% | 95.5 | 95.5 | 96.3 | 95.9 | 94.8 | 93.7 | 96.4 | 94.0 | 95.7 | 92.9 | 95.0 | 94.7 | 95.2 | 92.6 |
| BHA | LC | | | | | | | | | | | | | | |
| Content of | NLT 60% | 101.0 | 101.0 | 98.8 | 101.1 | 100.4 | 102.2 | 104.6 | 104.0 | 100.4 | 99.9 | 96.3 | 97.0 | 92.6 | 93.7 |
| EtOH | LC | | | | | | | | | | | | | | |
| Imp. A | NMT 1.2% | 0.06 | 0.06 | 0.10 | 0.10 | 0.12 | 0.13 | 0.19 | 0.19 | 0.23 | 0.22 | 0.29 | 0.25 | 0.33 | 0.32 |
| Imp. C | NMT 1.5% | 0.50 | 0.50 | 0.46 | 0.51 | 0.56 | 0.54 | 0.61 | 0.65 | 0.67 | 0.68 | 0.83 | 0.80 | 0.80 | 0.80 |
| Imp. F | NMT 0.2% | BLOQ | BLOQ | BLOQ | BLOQ | 0.02 | 0.02 | 0.02 | 0.02 | 0.02 | 0.02 | 0.02 | 0.02 | 0.02 | 0.03 |
| SMUI | NMT 0.2% | 0.08 | 0.08 | 0.09 | 0.08 | 0.08 | 0.08 | 0.09 | 0.09 | 0.11 | 0.11 | 0.17 | 0.17 | 0.19 | 0.19 |
| TI | NMT 2.0% | 0.25 | 0.25 | 0.35 | 0.35 | 0.48 | 0.50 | 0.63 | 0.64 | 0.68 | 0.68 | 0.92 | 0.83 | 1.02 | 1.00 |

Notes:
Description: Clear colorless to pale yellow solution (complies).
Density: Specification (1.205-1.255 g/mL); observed ≈1.24 g/mL through 12 M.
Abbreviations: LC (labelled content), NLT (not less than), NMT (not more than), BLOQ (below limit of quantification), SMUI (Single Maximum Unspecified Impurity), and TI (total impurities without Impurity C).

Based on the data presented in Table 8, Solution 3 is pharmaceutically acceptable for 24-months (or more) at when stored at 25° C.±2° C./60±5% RH. This data supports a shelf life for Solution 3 of about 24-months at least because the amount of Imp. A is NMT 1.2%. Additionally, Solution 3 may exhibit a longer shelf-life (e.g., 48-months) because a shelf-life regression analysis with respect to Impurity A for the upright bottles, resulted in a linear plot of % Imp. A=0.0106t+0.0725 (R²=0.973), which results in a % Imp. A of about 0.6% at t=48M. The data (inverted bottles) confirms that Solution 3 is pharmaceutically acceptable for 24-months (or more) when stored at 25° C.±2° C./60±5% RH.

Antimicrobial Effectiveness Testing showed that Solution 3 meets the criteria for antimicrobial effectiveness as per U.S. and European pharmacopoeias. For instance, Solution 3 shows no increase in any one of five organisms (viz., S. aureus (ATCC 6538), P. aeruginosa (ATCC 9027), E. coli (ATCC 8739), C. albicans (ATCC 10231), and A. brasiliensis (ATCC 16404) after storage at 25° C.±2° C./60±5% RH for 12-months.

Solution 4—Clopidogrel, 15 mg/mL

Batches (200 L, ≈249 kg) are manufactured with the compositional makeup shown in Table 9.

TABLE 9
Solution of Clopidogrel, 15 mg/mL, batch size of 200 L

| | Formulation ID | | |
|---|---|---|---|
| | Solution 4 (200 L ≈ 246 kg) | | |
| | Quantity | Quantity | Amount |
| Ingredients | (mg/mL) | (kg) | (% w/w) |
| Clopidogrel | 19.58 (≈15)²⁾ | 3.916 (≈3.00) | 1.59 (≈1.22) |
| bisulfate¹⁾ | | | |
| (clopidogrel eq.) | | | |
| Propylene Glycol | 70.0 | 14.0 | 5.69 |
| Ethanol | 33.18³⁾ | 6.636 | 2.70 |
| Butylated | 0.10⁴⁾ | 0.020 | 0.008 |
| Hydroxyanisole | | | |
| Sucralose | 1.0 | 0.200 | 0.081 |
| Frozen Peppermint | 0.5 | 0.100 | 0.041 |
| Flavor | | | |
| Glycerin | Q.S. to 1 mL⁵⁾ | Q.S. to ≈246 kg | 89.89 |

Notes
¹⁾19.58 mg/mL of clopidogrel bisulfate corresponds to about 15 mg/mL clopidogrel.
²⁾Final quantity to be dispensed after potency correction.
³⁾5% overage of ethanol providing ethanol content 0.042 mL EtOH/mL Solution (or about 4% v/v).
⁴⁾Final quantity to be dispensed after potency correction.
⁵⁾Target density of 1.230 g/mL with density specification of 1.205-1.255 g/mL.

### Manufacturing Processes

The oral pharmaceutical solution disclosed herein may be prepared by processes as described below.

Oral pharmaceutical solutions disclosed herein generally can be prepared by dissolving the drug and excipients in the vehicle and make up the solution to the appropriate volume. A process for preparing an oral pharmaceutical solution disclosed herein may comprise separately dissolving the drug and excipients or any combination thereof, and subsequently combining one or more of the solvated drugs or excipients. For instance, a process for preparing an oral pharmaceutical solution disclosed herein may comprise preparing a first mixture of propylene glycol and the vehicle in a first vessel, preparing a second mixture of ethanol and additional excipients in a second vessel, and combining the second mixture with the first mixture in the first vessel. Processes disclosed herein may further comprise adding clopidogrel or a pharmaceutically acceptable salt (e.g., clopidogrel bisulfate) to the premixture. An additional amount of vehicle and an additional excipient may be mixed into the premixture. A process disclosed herein may comprise adding vehicle to the mixture in a quantity sufficient to provide the clopidogrel or a pharmaceutically acceptable salt (e.g., clopidogrel bisulfate) in the desired concentration.

In one aspect, the oral pharmaceutical solution composition of clopidogrel or a pharmaceutically acceptable salt thereof may be prepared by a process comprising the steps of:
1) Adding a suitable amount of a vehicle to a vessel,
2) Optionally, adding a cosolvent to the vessel with stirring until a clear solution is obtained,
3) Adding an antioxidant to the mixture of step 2) with stirring until a clear solution is obtained,
4) Optionally adding a sweetener to the mixture of step 3) with stirring until a clear solution is obtained,
5) Adding clopidogrel or a pharmaceutically acceptable salt thereof to the mixture of step 4) with stirring until a clear solution is obtained,
6) Optionally adding a flavorant to the mixture of step 5) with stirring until the clear solution is obtained,
7) Making up the final volume with a quantity sufficient of the vehicle and stirring until a clear solution is obtained.

As per one embodiment, the oral pharmaceutical solution composition of clopidogrel or a pharmaceutically acceptable salt thereof may be prepared by the process comprising the steps of:
1) Adding a half quantity of the vehicle in a vessel,
2) Adding propylene glycol with stirring until a clear solution is obtained,
3) Adding ethanol to the mixture of step 2) with stirring until a clear solution is obtained,
4) Adding an antioxidant to the mixture of step 3) with stirring until a clear solution is obtained,
5) Optionally adding a sweetener to the mixture of step 4) with stirring until a clear solution is obtained,
6) Adding clopidogrel bisulfate to the mixture of step 5) with stirring until a clear solution is obtained,
7) Optionally adding a flavorant to the mixture of step 6) with stirring until the clear solution is obtained,
8) Making up the final volume with a quantity sufficient of the vehicle and stirring until a clear solution is obtained.

As per one specific embodiment, the oral pharmaceutical solution may be prepared by the process comprising the steps of:
1) Adding glycerin in a vessel,
2) Adding propylene glycol with stirring until a clear solution is obtained,
3) Adding ethanol to a second vessel,
4) Adding butylated hydroxyanisole to the mixture of step 3) with stirring until a clear solution is obtained,
5) Optionally adding sucralose to the mixture of step 4) with stirring until clear solution is obtained,
6) Combining the solution obtained in step 5) to the solution obtained in step 2),
7) Adding clopidogrel bisulfate to the mixture of step 6) with stirring until a clear solution is obtained,
8) Optionally adding a flavorant to the mixture of step 7) with stirring until a clear solution is obtained,
9) Making up the final volume of the solution with a quantity sufficient of glycerin and stirring until a clear solution is obtained.

After an oral pharmaceutical solution as disclosed herein is mixed, the oral pharmaceutical solution may be filtered and bottled for storage. For instance, the oral pharmaceutical solution prepared as described above may be filtered using a 40-micron polypropylene filter, and the filtrate may be added to a 150 mL amber glass bottle (with filling range of about 154 mL to about 158 mL), capped with 28 mm PP, EPE Wadded, child resistant closure, tamper evident (TE) ring.

### Practical Utility

The oral pharmaceutical solutions disclosed herein show practical utility with respect to their long-term stability and content uniformity.

The oral pharmaceutical solutions disclosed herein exhibit improved properties compared to the commercially marketed PLAVIX® solid dosages because the dose delivery method can be improved for patient populations that have difficulty swallowing. Oral pharmaceutical solutions disclosed herein also can be individualized to a greater extent and avoid detrimental attributes arising from low pH aqueous solutions. Oral pharmaceutical solutions disclosed herein also demonstrate improved stability compared to suspensions that require on-demand compounding. Oral pharmaceutical solutions disclosed herein demonstrate stability without refrigeration, and permissive to excursions and thermal cycling to maintain a suitable shelf-life of up to 24-months.

The oral pharmaceutical solutions disclosed herein are superior to Skillman's extemporaneously prepared suspensions (clopidogrel, 5 mg/mL), which require on-demand compounding, showed reduced clopidogrel stability, and reduced concentration of dose at 5 mg/mL.

The oral pharmaceutical solutions disclosed herein are superior to Rosemont's clopidogrel-containing composition at least because Rosemont's composition has a high amount of ethanol (e.g., 10% v/v (or 6.4% w/w)). Again, clopidogrel in combination with aspirin is used for long-term treatment of, for example, reducing risk of heart attack and/or stroke. And aspirin in combination with ethanol is contraindicated. Thus, Rosemont's clopidogrel-containing composition is pharmaceutically unacceptable at least because clopidogrel is used in combination with aspirin and aspirin is contraindicated with ethanol. It follows that Rosemont's clopidogrel-containing composition is contraindicated in combination with aspirin because of the high amount of ethanol. Further, a pharmaceutical composition having a high amount of alcohol is unacceptable for certain patient populations (e.g., pediatric patients).

Further, a pharmaceutically acceptable clopidogrel-containing solution disclosed herein reduces potentially undesirable side-effects (due to clopidogrel-aspirin co-administration), as well as the undesirable adverse effects arising from co-administering ethanol and aspirin.

An oral pharmaceutical solution disclosed herein has a clopidogrel content of 100±10% clopidogrel labeled content when stored for about W-months at 25±2° C. and 60±5% relative humidity, where W is 1, 3, 6, 9, 12, 18, 24, 30, 36, 42, or 48.

Additionally, an oral pharmaceutical solution disclosed herein has an amount of Impurity A of not more than about 1.2% w/w when stored for about X-months at 25±2° C. and 60±5% relative humidity, where X is 1, 3, 6, 9, 12, 18, 24, 30, 36, 42, or 48.

Moreover, an oral pharmaceutical solution contemplated herein has an amount of Impurity C of not more than about 1.5% w/w when stored for at least about Y-months at 25±2° C. and 60±5% relative humidity, where Y is 1, 3, 6, 9, 12, 18, 24, 30, 36, 42, or 48.

Further, an oral pharmaceutical solution contemplated herein has an amount of Impurity F of not more than about 0.2% w/w when stored for at least about Z-months at 25±2° C. and 60±5% relative humidity, where Z is 1, 3, 6, 9, 12, 18, 24, 30, 36, 42, or 48.

An oral pharmaceutical solution contemplated herein has a density of about 1.205 to about 1.255 g/mL (e.g., 1.230 g/mL) when stored for at least about Z-months at 25±2° C. and 60±5% relative humidity, where Z is 1, 3, 6, 9, 12, 18, 24, 30, 36, 42, or 48.

### CITED INFORMATION

21 C.F.R. 328.10 (Alcohol), 2013.
Allen, L. V., Clopidogrel 5 mg mL Oral Suspension, US Pharm. (2010) 35(5): 42-43.
Bayer (aspirin (acetylsalicylic acid), 300 mg) tablets Professional Information (2021) (“Bayer Information”).
Berge et al., Pharmaceutical Salts, J. Pharm. Science (1977) 66(1): 1-19 (“Berge”).
Engels et al., Standardization of compounded oral liquids for pediatric patients in Michigan, Am. J. Health-Syst Pharm, 73(13): 981-990 (“Engels”).
European Patent No. 1 105 102 B1, Composition pharmaceutique injectable a base d'un sel pharmaceeutiquement acceptable du clopidogrel ou de ticlopidine, published on Jan. 2, 2003 to Aleman et al. of Sanofi-Synthelabo (“Sanofi-Synthelabo”).
Li et al., Dosing of Clopidogrel for Platelet Inhibition in Infants and Young Children, Circulation (2008) 117(4): 553-559 (“Li”).
PCT Publication No. WO 00/10534 A1, Pharmaceutical composition for injection based on a pharmaceutically acceptable clopidogrel or ticlopidin salt, to Aleman et al. of Sanofi Aventis France.
PCT International Publication No. WO 2009/109741 A1, Clopidogrel solution for oral administration, to Ingrid Maria Soderberg of Rosemont Pharmaceuticals Ltd. (“Rosemont”).
PCT International Publication No. WO 2009/133455 A2, Pharmaceutical composition of clodipogrel, to Bhushan et al. of Cadila Heathcare Limited (“Cadila”).

PLAVIX® (clopidogrel bisulfate) tablets prescribing information as of May 17, 2019 (“PLAVIX® PI”).

ORA-PLUS® Product information (2010).

ORA-SWEET® Product information (2010).
Skillman et al., Stability of an extemporaneously prepared clopidogrel oral suspension, Am. J. Health-Syst Pharm, 67:559-561 (“Skillman”).
Rao et al., Design, development and evaluation of clopidogrel bisulfate floating tablets, Int J. Pharm. Investig. (2014) 4(1): 19-26 (“Rao”).
Reist et al., Very Slow Chiral Inversion of Clopidogrel in Rats: A Pharmacokinetic and Mechanistic Investigation, Drug. Metab. Disposition (2000) 28(12): 1405-1410 (“Reist”).
The United States Pharmacopeia (USP 28)| The National Formulary (NF 23), 2005 (“USP 28”).
U.S. Pat. No. 4,529,596, Thieno(3,2-c) pyridine derivatives, process for their preparation and their therapeutical use, to Aubert et al. of Sanofi SA (“Aubert”).
U.S. Pat. No. 4,847,265, Dextro-rotatory enantiomer of methyl alpha-5 (4,5,6,7-tetrahydro (3,2-c) thieno pyridyl) (2-chlorophenyl)-acetate and the pharmaceutical compositions containing it, to Badorc et al. (“Badorc”).
U.S. Pat. No. 5,576,328, Method for the secondary prevention of ischemic events, to Herbert et al. of Elf Sanofi.
U.S. Pat. No. 5,989,578, Associations of active principles containing clopidogrel and an antithrombotic agent, to Bernat et al. of Sanofi.
U.S. Pat. No. 6,284,277, Stable freeze-dried pharmaceutical formulation, to Bouloumie et al. of Sanofi-Synthelabo (“Bouloumie”).
U.S. Pat. No. 6,429,210, Polymorphic clopidogrel hydrogenesulphate form, to Bousquet et al. of Sanofi Aventis France.
U.S. Pat. No. 6,504,030, Polymorphic form of clopidogrel hydrogen sulphate, to Bousquet et al. of Sanofi Aventis France.
U.S. Pat. No. 6,635,763, Process to prepare clopidogrel, to Pandey et al of Cadilla Healthcare Ltd.
U.S. Pat. No. 6,737,411, Racemization and enantiomer separation of clopidogrel, to Valeriano et al. of Teva Pharmaceuticals USA, Inc.
U.S. Pat. No. 6,800,759, Racemization and enantiomer separation of clopidogrel, to Valeriano et al. of Teva Pharmaceuticals USA, Inc.
U.S. Pat. No. 6,858,734, Preparation of (S)-Clopidogrel and related compounds, to Richard A. Silva of Shasun Pharma Solutions Inc.
U.S. Pat. No. 8,343,995, Formulations containing clopidogrel and sulfoalkyl ether cyclodextrin and methods of use, to Mosher et al. of Cydex Pharmaceuticals, Inc. (“Mosher”).
Weathermon et al., Alcohol and Medical Interactions, Alcohol Research & Health (1999) 23(1): 40-54 (“Weathermon”).

Alternative embodiments, examples, and modifications which would be encompassed by the disclosure may be made by those skilled in the art, particularly considering the foregoing teachings. Further, the terminology used to describe the disclosure is intended to be in the nature of words of description rather than of limitation.

The subject matter of U.S. Provisional Patent Application No. 63/228,623, filed on Aug. 3, 2021, is incorporated by reference in its entirety. Additionally, the subject matter of the information cited herein is incorporated by reference in its entirety, to the extent necessary. If there is a difference in meaning between the incorporated terms and the terms disclosed herein, the meaning of the terms disclosed herein will control.

Those skilled in the art will also appreciate that various adaptations and modifications of the preferred and alternative embodiments described above can be configured without departing from the scope and spirit of the disclosure. Therefore, it is to be understood that, within the scope of the appended claims, the disclosure may be practiced other than as specifically described herein.

1. An oral pharmaceutical solution, comprising:
about 0.5 mg/mL to about 120 mg/mL of clopidogrel or a pharmaceutically acceptable salt thereof;
about 35 mg/mL to about 200 mg/mL propylene glycol;
one or more pharmaceutically acceptable excipients; and
a vehicle comprising glycerin;
wherein the oral pharmaceutical solution has an amount of water of less than or equal to 5% w/w and an amount of ethanol less than or equal to 6% w/w.

2. The oral pharmaceutical solution of claim 1 comprising clopidogrel or a pharmaceutically acceptable salt thereof in an amount of from about 5 mg/mL to 35 mg/mL.

3. The oral pharmaceutical solution of claim 1 comprising clopidogrel or a pharmaceutically acceptable salt thereof in an amount of from about 10 mg/mL to about 25 mg/mL.

4. The oral pharmaceutical solution of claim 1 comprising clopidogrel or a pharmaceutically acceptable salt thereof in an amount of from about 13 mg/mL to about 17 mg/mL.

5. The oral pharmaceutical solution of claim 1 comprising clopidogrel or a pharmaceutically acceptable salt thereof in an amount of about 15 mg/mL.

6. The oral pharmaceutical solution of claim 1 comprising clopidogrel bisulfate in an amount of from about 19 mg/mL to about 20 mg/mL.

7. The oral pharmaceutical solution of claim 1 comprising propylene glycol in an amount of from about 70 mg/mL to about 200 mg/mL.

8. The oral pharmaceutical solution of claim 1 comprising propylene glycol in an amount of from about 70 mg/mL to about 100 mg/mL.

9. The oral pharmaceutical solution of claim 1 comprising propylene glycol in an amount of about 70 mg/mL.

10. The oral pharmaceutical solution of claim 1, wherein the one or more pharmaceutically acceptable excipients comprises ethanol in an amount of from about 16 mg/mL to about 33 mg/mL.

11. The oral pharmaceutical solution of claim 1, wherein the one or more pharmaceutically acceptable excipients comprises ethanol in an amount of from about 16 mg/mL to about 32 mg/mL.

12. The oral pharmaceutical solution of claim 1, wherein the one or more pharmaceutically acceptable excipients comprises ethanol in an amount of from about 32 to about 33 mg/mL.

13. The oral pharmaceutical solution of claim 1, wherein the oral pharmaceutical solution has an amount of ethanol less than or equal to about 2.7% w/w.

14. The oral pharmaceutical solution of claim 1, wherein the one or more pharmaceutically acceptable excipients comprises an antioxidant, a sweetener, a flavorant, or a combination thereof.

15. The oral pharmaceutical solution of claim 14, wherein the antioxidant comprises ascorbyl palmitate, butylated hydroxyanisole, butylated hydroxytoluene, erythorbic acid, ethyl oleate, methionine, monothioglycerol, propyl gallate, sodium ascorbate, thymol, tocopherol, vitamin E, and vitamin E polyethylene glycol succinate, or a combination thereof.

16. The oral pharmaceutical solution of claim 14, wherein the antioxidant comprises butylated hydroxyanisole.

17. The oral pharmaceutical solution of claim 1, wherein the one or more pharmaceutically acceptable excipients comprises an antioxidant comprising butylated hydroxyanisole in an amount of from about 0.06 mg/mL to about 0.14 mg/mL.

18. The oral pharmaceutical solution of claim 14, wherein the sweetener comprises glucose, sucralose, trehalose, fructose, xylose, dextrose, galactose, tagatose, maltose, sucrose, glycerol, dulcitol, mannitol, lactitol, sorbitol, xylitol, saccharine, cyclamate, aspartame, or acesulfame, dulcin or ammonium glycyrrhizinate, alitame, inulin, isomalt, neohesperidin dihydrochalcone, thaumatin, or a combination thereof.

19. The oral pharmaceutical solution of claim 1, wherein the one or more pharmaceutically acceptable excipients comprises a sweetener in an amount of from about 0.5 mg/mL to about 1.5 mg/mL.

20. The oral pharmaceutical solution of claim 14, wherein the flavorant comprises anise oil, apple, apricot, banana, bay oil, cassia oil, cedar leaf oil, cherry, cinnamon oil, citrus oil, clove oil, eucalyptus, frozen peppermint, grape, grapefruit, lemon, lime, mixed berry, oil of bitter almonds, oil of nutmeg, oil of sage, oil of wintergreen, orange, peach, pear, peppermint, pineapple, plum, raspberry, strawberry, thyme oil, Tutti Frutti flavor, vanilla, or a combination thereof.

21. The oral pharmaceutical solution of claim 1, wherein the one or more pharmaceutically acceptable excipients comprises a flavorant in an amount of about 0.1 mg/mL to about 1.0 mg/mL.

22. A dosage dispensing unit comprising the oral pharmaceutical solution of claim 1.

23. The dosage dispensing unit of claim 22, wherein the oral solution stored for about 24 months at 25° C.±2° C. and 60±5% relative humidity has: (i) a clopidogrel content of 100±10%, (ii) less than 1.2% Impurity A, (iii) less than 1.5% Impurity C, (iv) less than 0.2% Impurity F, or a combination thereof.

24. A method for the treatment of a condition, which comprises administering to a patient in need thereof a therapeutically effective amount of the oral pharmaceutical solution of claim 1, optionally, in combination with aspirin, wherein the condition comprises acute coronary syndrome, myocardial infarction, stroke, peripheral arterial disease, or a combination thereof.

25. A process for preparing the oral pharmaceutical solution of claim 1, said process comprising combining the clopidogrel or a pharmaceutically acceptable salt, the propylene glycol, the one or more pharmaceutically acceptable excipients, and the vehicle comprising glycerin.

26. An oral pharmaceutical solution comprising:
about 15 mg/mL of clopidogrel or a pharmaceutically acceptable salt thereof;
about 0.09 to about 0.11 mg/mL of an antioxidant;
about 50 mg/mL to about 100 mg/mL of propylene glycol;
about 32 mg/mL to about 33 mg/mL of ethanol;
one or more pharmaceutically acceptable excipients comprising a sweetener, a flavorant, or a combination thereof; and
a vehicle comprising glycerin;
wherein the oral pharmaceutical solution has an amount of water of less than or equal to 5% w/w.

27. A dosage dispensing unit comprising the oral pharmaceutical solution of claim 1.

28. The dosage dispensing unit of claim 27, wherein the oral solution stored for about 24 months at 25° C.±2° C. and 60±5% relative humidity has: (i) a clopidogrel content of 100±10%, (ii) less than 1.2% Impurity A, (iii) less than 1.5% Impurity C, (iv) less than 0.2% Impurity F, and a combination thereof.

29. A method for the treatment of a condition, which comprises administering to a patient in need thereof a therapeutically effective amount of the oral pharmaceutical solution of claim 26, optionally, in combination with aspirin, wherein the condition comprises acute coronary syndrome, myocardial infarction, stroke, peripheral arterial disease, or a combination thereof.

30. A process for preparing the oral pharmaceutical solution of claim 26, said process comprising combining the clopidogrel or a pharmaceutically acceptable salt, the antioxidant, the propylene glycol, the ethanol, the one or more pharmaceutically acceptable excipients, and the vehicle comprising glycerin.
